SEQUENCE LISTING (46 sequences)

SEQ ID NO: 1 - DNA, 16 bp, Artificial sequence
  Synthetic oligonucleotide
ggctactacgccgtca

SEQ ID NO: 2 - DNA, 16 bp, Artificial sequence
  Synthetic oligonucleotide
gcattctaatagcagc

SEQ ID NO: 3 - DNA, 23 bp, Artificial sequence
  Primer
tgggttagagaaggcgtgtactg

SEQ ID NO: 4 - DNA, 18 bp, Artificial sequence
  Primer
tcagcggcaactgggaaa

SEQ ID NO: 5 - DNA, 25 bp, Artificial sequence
  Probe
cgttggcacgacaccttcagggact

SEQ ID NO: 6 - DNA, 22 bp, Artificial sequence
  Primer
ttcagtaaccagtacaaagcca

SEQ ID NO: 7 - DNA, 20 bp, Artificial sequence
  Primer
gtagaaggccacaccaagag

SEQ ID NO: 8 - DNA, 24 bp, Artificial sequence
  Probe
ctgtcgtccaccatcacctccttg

SEQ ID NO: 9 - DNA, 22 bp, Artificial sequence
  Primer
ccagattgtcctaaagcagttg

SEQ ID NO: 10 - DNA, 21 bp, Artificial sequence
  Primer
accaatctccaacacatcagt

SEQ ID NO: 11 - DNA, 27 bp, Artificial sequence
  Probe
tggtgaacctcagtgaatgtatggacc

SEQ ID NO: 12 - DNA, 19 bp, Artificial sequence
  Primer
ccaagtcccaaacactgct

SEQ ID NO: 13 - DNA, 22 bp, Artificial sequence
  Primer
ccaggtagacagaattagccat

SEQ ID NO: 14 - DNA, 24 bp, Artificial sequence
  Probe
ctggaagtcatacagcgccacgat

SEQ ID NO: 15 - DNA, 24 bp, Artificial sequence
  Primer
gacgaattccaacttgctttactc

SEQ ID NO: 16 - DNA, 23 bp, Artificial sequence
  Primer
gtcagttaccaacgtcaaatagc

SEQ ID NO: 17 - DNA, 32 bp, Artificial sequence
  Probe
tggttctgcttctctttcagttatctcatcct

SEQ ID NO: 18 - DNA, 20 bp, Artificial sequence
  Primer
gtcttcaatgccaccttcca

SEQ ID NO: 19 - DNA, 22 bp, Artificial sequence
  Primer
gcattgatttcattccactcca

SEQ ID NO: 20 - DNA, 22 bp, Artificial sequence
  Probe
ttgtgccaaactgtccgctgtg

SEQ ID NO: 21 - DNA, 23 bp, Artificial sequence
  Primer
gaagtgacggaccgttctatgac

SEQ ID NO: 22 - DNA, 18 bp, Artificial sequence
  Primer
ggaggaacgaggcgttga

SEQ ID NO: 23 - DNA, 26 bp, Artificial sequence
  Probe
tggaaacgaccttctacgacgatgcc

SEQ ID NO: 24 - DNA, 20 bp, Artificial sequence
  Synthetic oligonucleotide
ccttccctgaaggttcctcc

SEQ ID NO: 25 - DNA, 20 bp, Artificial sequence
  Synthetic oligonucleotide
gggtcagctgccaatgctag

SEQ ID NO: 26 - DNA, 20 bp, Artificial sequence
  Synthetic oligonucleotide
gcttcagtcatgacttcctt

SEQ ID NO: 27 - DNA, 20 bp, Artificial sequence
  Primer
tgacaacgacaccgtgtcct

SEQ ID NO: 28 - DNA, 19 bp, Artificial sequence
  Primer
atgcgacttgtcaggctgg

SEQ ID NO: 29 - DNA, 23 bp, Artificial sequence
  Probe
cgtggagaaccgcagcctccatt

SEQ ID NO: 30 - DNA, 20 bp, Artificial sequence
  Synthetic oligonucleotide
ctgctagcctctggatttga

SEQ ID NO: 31 - DNA, 20 bp, Artificial sequence
  Synthetic oligonucleotide
ggcatatgcagataatgttc

SEQ ID NO: 32 - DNA, 21 bp, Artificial sequence
  Primer
aaagcaaggtctccccacaag

SEQ ID NO: 33 - DNA, 24 bp, Artificial sequence
  Primer
tgaagggtctgtgctagatcaaaa

SEQ ID NO: 34 - DNA, 19 bp, Artificial sequence
  Probe
tgccacatcgccaccccgt

SEQ ID NO: 35 - DNA, 17 bp, Artificial sequence
  Primer
gccatggagcgctttgg

SEQ ID NO: 36 - DNA, 22 bp, Artificial sequence
  Primer
tccacagtcagcaatggtgatc

SEQ ID NO: 37 - DNA, 25 bp, Artificial sequence
  Probe
tccaggaatggcaagaccagcaaga

SEQ ID NO: 38 - DNA, 20 bp, Artificial sequence
  Primer
caccattgctgactgtggac

SEQ ID NO: 39 - DNA, 20 bp, Artificial sequence
  Primer
actctgcaatccagctaggc

SEQ ID NO: 40 - DNA, 50 bp, Artificial sequence
  Probe
ttctgtagctcaggagagcacccctccaccccatttgctcgcagtatcct

SEQ ID NO: 41 - DNA, 20 bp, Artificial sequence
  Synthetic oligonucleotide
ccaggctggttatgactcag

SEQ ID NO: 42 - DNA, 16 bp, Artificial sequence
  Synthetic oligonucleotide
gaaattcattcttcca

SEQ ID NO: 43 - DNA, 16 bp, Artificial sequence
  Synthetic oligonucleotide
gtatgctgtggctttt

SEQ ID NO: 44 - DNA, 26 bp, Artificial sequence
  Primer
aatggctaagtgaagatgacaatcat

SEQ ID NO: 45 - DNA, 25 bp, Artificial sequence
  Primer
tgcacatatcattacaccagttcgt

SEQ ID NO: 46 - DNA, 30 bp, Artificial sequence
  Probe
ttgcagcaattcactgtaaagctggaaagg

(19) United States
(12) Patent Application Publication
(10) Pub. No.: US 2021/0130826 A1
(43) Pub. Date: May 6, 2021

(54) METHODS OF MODULATING ANTISENSE ACTIVITY

(71) Applicant: IONIS PHARMACEUTICALS, INC., Carlsbad, CA (US)

(72) Inventors: Joseph OCHABA, JR., Oceanside, CA (US); Mariam AGHAJAN, San Diego, CA (US)

(73) Assignee: Ionis Pharmaceuticals, Inc., Carlsbad, CA (US)

(21) Appl. No.: 17/041,677

(22) PCT Filed: Apr. 4, 2019
(86) PCT No.: PCT/US2019/025734

§ 371 Date: Sep. 25, 2020

Related U.S. Application Data
(60) Provisional application No. 62/734,827, filed on Sep. 21, 2018.
(60) Provisional application No. 62/671,274, filed on May 14, 2018.
(60) Provisional application No. 62/654,077, filed on Apr. 6, 2018.

(51) Int. Cl. C12N 15/113

(52) U.S. Cl. CPC C12N 15/113; C12N 2310/341; C12N 2310/313

(57) ABSTRACT

Disclosed herein are methods for increasing antisense oligonucleotide activity in a cell by modulating autophagy of the cell. In certain embodiments, a compound comprising an antisense oligonucleotide is co-administered to a subject with an autophagy modulator.

### SEQUENCE LISTING

The present application is being filed along with a Sequence Listing in electronic format. The Sequence Listing is provided as a file entitled CORE0149WO3SEQ_ST25.txt, created Apr. 1, 2019, which is 12 Kb in size. The information in the electronic format of the sequence listing is incorporated herein by reference in its entirety.

### BACKGROUND

Autophagy is a process in which cellular components are destroyed by being taken up in vesicles along with a portion of a cell's cytoplasm and subsequently delivered to lysosomes for degradation and recycling. This process is part of normal cell homeostasis and is involved in many physiologic functions, including immunity, cell survival, development, and differentiation.

The mechanisms by which antisense compounds, including antisense oligonucleotides, are taken up into cells in the absence of transfection reagents and ultimately reach their target nucleic acids are not fully understood. Uptake pathways of antisense compounds, such as antisense oligonucleotides, that result in pharmacological effects are referred to as productive uptake pathways.

### SUMMARY OF THE INVENTION

The present disclosure provides methods of increasing antisense activity by modulating autophagy. The methods provided herein comprise contacting a cell with an antisense compound and contacting a cell with an autophagy modulator. In certain embodiments, the autophagy modulation is activation of autophagy. In certain embodiments, the autophagy modulation is blocking the fusion of autophagosomes with lysosomes. In certain embodiments, the autophagy modulation increases antisense activity by increasing productive uptake of the antisense compound. In certain embodiments, the antisense activity of the antisense compound is reduction of the level of a target nucleic acid. In certain embodiments, the antisense activity of the antisense compound is splicing modulation of a target nucleic acid. In certain embodiments, the antisense activity of the antisense compound is increase of the level of a target nucleic acid. In certain embodiments, the methods herein comprising autophagy modulation result in an extent of antisense activity that is greater than the extent of antisense activity that occurs when autophagy is not modulated.

The working examples provided herein demonstrate that autophagy can increase antisense activity. This is demonstrated with many types of autophagy modulators, many cell types, and a variety of antisense oligonucleotides chemically modified in multiple ways. Thus, autophagy modulation that results in increased antisense activity may occur at one or more steps of the autophagy pathway. For example, some autophagy modulators that increase antisense activity increase the rate of autophagosome formation and/or the number of autophagosomes present in the cell at a given time after autophagy modulation. Autophagosome formation involves multiple steps, including autophagosome nucleation, elongation, sequestration of cytoplasmic contents, and completed formation of an intact autophagosome. See, e.g., Yu et al. Autophagy. September 21: 1-9 (2017) Epub ahead of print. Antisense compounds in productive uptake pathways may be contained in some autophagosomes and/or may shuttle between autophagosomes and/or other intracellular vesicles. Accordingly, autophagosomes can fuse temporarily or permanently with endosomes. Eventually, autophagosomes fuse with lysosomes to form autolysosomes. In some cases, autolysosomes are acidified and their contents are destroyed. In some cases, autolysosomes break apart and revert to separate autophagosomes and lysosomes. Some autophagy modulators that increase antisense activity block the fusion of autophagosomes with lysosomes. In some such cases, antisense compounds that were headed for degradation in an unproductive uptake pathway may be redirected to a productive uptake pathway.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising 2′-H(H) ribosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-fluoro” or “2′-F” means a 2′-F in place of the 2′—OH group of a ribosyl ring of a sugar moiety.

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

As used herein, “antisense activity” means any detectable and/or measurable change attributable to the hybridization of an antisense compound to its target nucleic acid. In certain embodiments, antisense activity is a decrease in the amount or expression of a target nucleic acid compared to target nucleic acid levels in the absence of the antisense compound. In certain embodiments, antisense activity is an increase in the amount or expression of a target nucleic acid compared to target nucleic acid levels in the absence of the antisense compound. In certain embodiments, antisense activity is a modulation of splicing a target nucleic acid compared to target nucleic acid splicing in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a method means improvement in at least one symptom of a disease or condition and/or measurable outcome relative to the same symptom or measurable outcome in the absence of or prior to performing the method. In certain embodiments, amelioration comprises a reduction in severity of a symptom, reduction in frequency of a symptom, delayed onset of the disease or condition, slowing of progression of the disease or condition, or a combination thereof.

As used herein, “ATP-competitive inhibitor” is a kinase inhibitor that binds to the ATP binding site of one or more kinases. When an ATP-competitive inhibitor is bound to the ATP binding site of a kinase, ATP cannot bind to the same site.

As used herein, “autophagy activation” means a modulation of autophagy in which the perturbation results in an increase of one or more steps or components of autophagy relative to the level of autophagy that occurs in the absence of the autophagy activation. The level of autophagy that occurs in the absence of the autophagy activation is that which is observed under conditions that are otherwise the same except that an autophagy activator is not present. In certain embodiments, an autophagy activator” is a compound or composition that activates autophagy in a cell after contacting the cell. In certain embodiments, an autophagy activator is a condition that activates autophagy. In certain embodiments, the condition that activates autophagy is fasting. In certain embodiments, the condition that activates autophagy is nutrient restriction (e.g., ketogenic diet).

As used herein, “autophagosome formation” means a process that begins with nucleation of a phagophore and ends with completed formation of an intact autophagosome.

As used here, “autophagy modulation” means a perturbation of activity, size, amount, and/or the rate of a step or component in autophagy. Steps in autophagy include phagophore nucleation, elongation, sequestration of cytoplasmic components, autophagosome formation, fusion of autophagosome to endosome, fusion of autophagosome to lysosome, and degradation of the components of the autolysosome. Components in autophagy include proteins and other molecules that contribute to carrying out the steps of autophagy. As used herein, an “autophagy modulator” is a compound or composition that modulates autophagy in a cell after contacting the cell. Processes that affect autophagy that do not involve contacting a cell with a compound or composition, such as fasting or nutrient restriction, are not autophagy modulators.

As used herein, “autophagic vesicle” means a double membraned vesicle that is physically associated with LC3-II.

As used herein, “basal” or “basal level” means the level or amount of a process observed in the absence of a particular stimulus. For example, a basal level of autophagy is the level of autophagy observed in the absence of an autophagy modulator or a stimulus that modulates autophagy, such as fasting.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cEt” or “constrained ethyl” means a ribosyl bicyclic sugar moiety wherein the second ring of the bicyclic sugar is formed via a bridge connecting the 4′-carbon and the 2′-carbon, wherein the bridge has the formula 4′-CH(CH₃)—O-2′, and wherein the methyl group of the bridge is in the S configuration.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. The term, “complementary nucleobases,” means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “COPB2” means coatomer protein complex subunit beta 2. COPB2 is encoded by the COPB2 gene.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” in reference to a gene or the product of a gene means the amount of a nucleic acid or protein that is encoded from a gene or encoded from a product of a gene. A change in expression, such as inhibition of expression, may be either a change to an encoding step itself (e.g., transcription or translation) or a change to the level of the encoded nucleic acid or protein, independent of any effects on the encoding step.

As used herein, “fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

As used herein, “gapmer” means an antisense oligonucleotide comprising an internal “gap” region having a plurality of nucleosides that support RNase H cleavage positioned between external “wing” regions having one or more nucleosides, wherein the nucleosides comprising the internal gap region are chemically distinct from the terminal wing nucleosides of the external wing regions.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting” or “inhibition” refers to a partial or complete reduction. For example, inhibiting the expression of a target nucleic acid means a partial or complete reduction of expression of the nucleic acid, e.g., a reduction in the amount of protein produced from the target nucleic acid, and does not necessarily indicate a total elimination of the protein or target nucleic acid. “Selective inhibition” is inhibition that occurs to a significantly greater extent for at least one identified target than for at least one other identified target. For example, a kinase inhibitor that is selective for kinase X over kinase Y inhibits kinase X to a significantly greater extent than kinase Y.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage. Modified internucleoside linkages include linkages that comprise abasic nucleosides.

As used herein, “LC3” means microtubule-associated protein 1A/1B-light chain 3. The LC3 protein is encoded by the MAP1LC3A gene. LC3-I is a cytosolic form of LC3. LC3-II is an LC-3-phosphatidylethanolamine conjugate.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a 2′-OCH₂CH₂OCH₃ group in place of the 2′—OH group of a ribosyl ring of a sugar moiety.

As used herein, “motif” means the pattern of unmodified and/or modified sugar moieties, nucleobases, and/or internucleoside linkages, in an oligonucleotide.

As used herein, “mTor” means mammalian target of rapamycin. mTor is a protein kinase that is encoded by the MTOR gene.

As used herein, “naturally occurring” means found in nature.

As used herein, “non-bicyclic modified sugar” or “non-bicyclic modified sugar moiety” means a modified sugar moiety that comprises a modification, such as a substituent, that does not form a bridge between two atoms of the sugar to form a second ring.

As used here, “nucleic acid delivery vehicle” means a cationic polymer, liposome, and/or nanoparticle containing composition used in combination with a nucleic acid in order to promote uptake of the nucleic acid by a cell. Nucleic acid delivery vehicles include but are not limited to commercially available transfection reagents.

As used herein, “nucleobase” means a naturally occurring nucleobase or a modified nucleobase. As used herein an “unmodified nucleobase” is adenine (A), thymine (T), cytosine (C), uracil (U), and guanine (G). As used herein, a modified nucleobase is a group of atoms capable of pairing with at least one naturally occurring nucleobase. A universal base is a nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group. As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

As used herein, “rapalog” means a derivative of the molecule, rapamycin.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound and/or antisense oligonucleotide means an oligomeric compound that is not paired with an additional oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex.

As used herein, “small molecule” means a molecule having a molecular weight equal to or less than 950 Daltons.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) moiety, as found in DNA (an “unmodified DNA sugar moiety”). As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” mean a nucleic acid that an antisense compound affects via hybridization to the target.

As used herein, “terminal group” means a chemical group or group of atoms that is covalently linked to a terminus of an oligonucleotide.

### Certain Effects of Autophagy Modulation on Antisense Activity

In order for certain antisense compounds, such as certain single-stranded antisense oligonucleotides, to exhibit antisense activity inside a cell, they must first enter the cell in a way that ultimately facilitates contact with a target nucleic acid. This is particularly important for antisense compounds that are used in the absence of any carrier, nanoparticle, or transfection reagent. Antisense compounds that enter an unproductive uptake pathway do not exhibit antisense activity. Antisense compounds that enter a productive uptake pathway may exhibit antisense activity. At least some productive uptake pathways include one or more steps in which the antisense compound is located inside of an intracellular vesicle. In such cases, the antisense compound must be released from the vesicle in order to reach its target nucleic acid. Modulating pathways that involve vesicular trafficking may increase antisense compound release or escape from vesicles and/or other steps in productive uptake pathways, ultimately leading to increased antisense activity.

Modulating autophagy can increase antisense activity. The autophagy modulation that results in increased antisense activity may occur at one or more steps of the autophagy pathway. For example, some autophagy modulators that increase antisense activity increase the rate of autophagosome formation and/or the number of autophagosomes present in the cell at a given time after autophagy modulation. Autophagosome formation involves multiple steps, including autophagosome nucleation, elongation, sequestration of cytoplasmic contents, and completed formation of an intact autophagosome. See, e.g., Yu et al. Autophagy. September 21: 1-9 (2017) Epub ahead of print. Antisense compounds in productive uptake pathways may be contained in some autophagosomes and/or may shuttle between autophagosomes and/or other intracellular vesicles. Accordingly, autophagosomes can fuse temporarily or permanently with endosomes. Eventually, autophagosomes fuse with lysosomes to form autolysosomes. In some cases, autolysosomes are acidified and their contents are destroyed. In some cases, autolysosomes break apart and revert to separate autophagosomes and lysosomes. Some autophagy modulators that increase antisense activity block the fusion of autophagosomes with lysosomes. In some such cases, antisense compounds that were headed for degradation in an unproductive uptake pathway may be redirected to a productive uptake pathway.

In certain embodiments, autophagy modulators have a specific effect on autophagy. In certain embodiments, autophagy modulators have more than one effect on autophagy. In certain embodiments, autophagy modulators have effects unrelated to autophagy as well as one or more effects on autophagy. The types of effects an autophagy modulator has on a cell may depend on multiple factors, including cell type, basal level of autophagy, and duration of exposure to the autophagy modulator. See, for example, Luu and Luty. Response Profiles of Known Autophagy-Modulators Across Multiple Cell Lines. Enzo Life Sciences Application Note.

### Certain Embodiments

The present disclosure includes but is not limited to the following embodiments:

1. A method comprising activating autophagy of a cell; and contacting the cell with an antisense compound comprising an antisense oligonucleotide, wherein the nucleobase sequence of the antisense oligonucleotide is complementary to a target nucleic acid.
2. The method of embodiment 1, wherein activating autophagy does not comprise contacting the cell with a nucleic acid delivery vehicle.
3. The method of embodiment 1 or 2, wherein the cell is not contacted with a nucleic acid delivery vehicle.
4. The method of any of embodiments 1-3 wherein an amount or expression of the target nucleic acid in the cell is modified.
5. The method of any of embodiments 1-4, wherein the amount or expression of the target nucleic acid is reduced.
6. The method of any of embodiments 1-3, wherein the target nucleic acid is a pre-mRNA, and the splicing of the target pre-mRNA is modulated.
7. The method of any of embodiments 1-3, wherein the amount or expression of the target nucleic acid is increased.
8. The method of embodiment 4, wherein the amount or expression is modified relative to a cell that is not contacted with the antisense compound and wherein autophagy of the cell is not activated.
9. The method of embodiment 4 or 5, wherein the expression or amount of the target nucleic acid is reduced to a greater extent than the extent of reduction of the expression or amount of the target nucleic acid that occurs in the absence of the autophagy modulator.
10. The method of embodiment 6, wherein the splicing of the target pre-mRNA is modulated to a greater extent than the extent of splicing modulation of the target pre-mRNA that occurs in the absence of the autophagy modulator.
11. The method of embodiment 7 or 8, wherein the expression or amount of the target nucleic acid is increased to a greater extent than the extent of increase of the expression or amount of the target nucleic acid that occurs in the absence of the autophagy modulator.
12. The method of any of embodiments 1-11, wherein the nucleobase sequence of the antisense oligonucleotide is at least 80% complementary to the target nucleic acid.
13. The method of any of embodiments 1-11, wherein the nucleobase sequence of the antisense oligonucleotide is at least 85% complementary to the target nucleic acid.
14. The method of any of embodiments 1-11, wherein the nucleobase sequence of the antisense oligonucleotide is at least 90% complementary to the target nucleic acid.
15. The method of any of embodiments 1-11, wherein the nucleobase sequence of the antisense oligonucleotide is at least 95% complementary to the target nucleic acid.
16. The method of any of embodiments 1-11, wherein the nucleobase sequence of the antisense oligonucleotide is 100% complementary to the target nucleic acid.
17. The method of any of embodiments 1-16, wherein the antisense oligonucleotide is a modified oligonucleotide.
18. The method of embodiment 17, wherein the modified oligonucleotide is a gapmer.
19. The method of embodiment 17 or 18, wherein the antisense oligonucleotide comprises at least one modified internucleoside linkage.
20. The method of embodiment 19, wherein the at least one modified internucleoside linkage is a phosphorothioate internucleoside linkage.
21. The method of embodiment 19, wherein all of the internucleoside linkages of the antisense oligonucleotide are modified internucleoside linkages.
22. The method of embodiment 21, wherein all of the internucleoside linkages of the antisense oligonucleotide are phosphorothioate internucleoside linkages.
23. The method of embodiment 20, wherein all of the internucleoside linkages of the antisense oligonucleotide are selected from phosphorothioate and phosphate internucleoside linkages.
24. The method of any of embodiments 17-23, wherein the antisense oligonucleotide comprises at least one modified sugar moiety.
25. The method of embodiment 24, wherein the at least one modified sugar moiety comprises a 2′-MOE, 2′-O-methyl, cEt, or LNA modification.
26. The method of embodiment 24 or 25, wherein the antisense oligonucleotide comprises at least two modified sugar moieties.
27. The method of embodiment 26, wherein the at least two modified sugar moieties are the same.
28. The method of embodiment 26, wherein the at least two modified sugar moieties are different.
29. The method of embodiment 26 or 27, wherein all of the modified sugar moieties of the antisense oligonucleotide are the same.
30. The method of embodiment 28, wherein each of the modified sugar moieties of the antisense oligonucleotide are independently selected from among modified sugar moieties comprising a 2′-MOE, 2′-O-methyl, cEt, or LNA modification.
31. The method of any of embodiments 24-30, wherein every nucleoside of the antisense oligonucleotide comprises a modified sugar moiety.
32. The method of any of embodiments 17-31, wherein the antisense oligonucleotide comprises at least one modified nucleobase.
33. The method of embodiment 32, wherein the at least one modified nucleobase is 5-methylcytosine.
34. The method of any of embodiments 1-33, wherein each nucleobase of the antisense oligonucleotide is independently selected from among adenine, thymine, guanine, cytosine, and 5-methylcytosine.
35. The method of any of embodiments 1-34, wherein the antisense compound is single-stranded.
36. The method of embodiment 35, wherein the antisense compound consists of a conjugate group and the antisense oligonucleotide.
37. The method of embodiment 35, wherein the antisense compound consists of the antisense oligonucleotide.
38. The method of any of embodiments 1-37, wherein the cell is a cancer cell.
39. The method of any of embodiments 1-37, wherein the cell is a not a cancer cell.
40. The method of any of embodiments 1-39, wherein the cell is in an animal.
41. The method of embodiment 40, wherein the animal is a human subject.
42. The method of embodiment 41 comprising administering the autophagy modulator and the antisense compound to the human subject.
43. The method of embodiment 42, wherein the human subject has a disease or condition that is ameliorated or treated by the administration of the antisense compound.
44. The method of embodiment 43, wherein the disease or condition is cancer.
45. The method of embodiment 43, wherein the disease or condition is progeria.
46. The method of any of embodiments 42-45, wherein the antisense compound and the autophagy modulator are administered simultaneously.
47. The method of any of embodiments 42-45, wherein the antisense compound and the autophagy modulator are administered sequentially.
48. The method of embodiment 47, wherein the antisense compound is administered before the autophagy modulator.
49. The method of embodiment 47, wherein the antisense compound is administered after the autophagy modulator.
50. The method of any of embodiments 1-49, wherein the autophagy modulator is a small molecule.
51. The method of any of embodiments 1-50, wherein the autophagy modulator is an autophagy activator.
52. The method of embodiment 51, wherein the autophagy activator increases autophagosome formation relative to that expected at a basal level of autophagy.
53. The method of embodiment 51, wherein the autophagy activator increases autophagosome nucleation and/or autophagosome elongation relative to that expected at a basal level of autophagy.
54. The method of any of embodiments 51-53, wherein the autophagy activator increases the number of autophagic vesicles in the cell relative to that expected at a basal level of autophagy.
55. The method of any of embodiments 51-53, wherein the autophagy activator increases the number of autophagosomes in the cell relative to that expected at a basal level of autophagy.
56. The method of any of embodiments 51-55, wherein the autophagy activator is an mTor inhibitor.
57. The method of any of embodiments 51-55, wherein the autophagy activator is capable of inhibiting mTor in the cell.
58. The method of embodiment 56 or 57, wherein the autophagy activator is rapamycin.
59. The method of embodiment 56 or 57, wherein the autophagy activator is a rapalog.
60. The method of embodiment 59, wherein the rapalog is temsirolimus.
61. The method of embodiment 59, wherein the rapalog is everolimus.
62. The method of embodiment 59, wherein the rapalog is ridaforolimus.
63. The method of embodiment 56 or 57, wherein the autophagy activator is an ATP-competitive mTor inhibitor.
64. The method of embodiment 56, 57, or 63, wherein the autophagy activator selectively inhibits mTor over other kinases.
65. The method of embodiment 56, 57, or 63, wherein the autophagy activator selectively inhibits mTor over PI3 kinase.
66. The method of embodiment 56, 57, or 63, wherein the autophagy activator inhibits mTor and at least one other kinase.
67. The method of any of embodiments 56, 57, or 63-66, wherein the autophagy activator is not a PI3 kinase inhibitor.
68. The method of embodiment 63, wherein the ATP-competitive mTor inhibitor is OSI-027.
69. The method of embodiment 63, wherein the ATP-competitive mTor inhibitor is AZD8055.
70. The method of embodiment 63, wherein the ATP-competitive mTor inhibitor is AZD2014.
71. The method of embodiment 63, wherein the ATP-competitive mTor inhibitor is INK128.
72. The method of embodiment 66, wherein the autophagy activator is PI-103.
73. The method of embodiment 56, 57, 64, 65, or 67, wherein the autophagy activator is PP242.
74. The method of any of embodiments 51-55, wherein the autophagy activator increases expression of LC3.
75. The method of any of embodiments 51-55 or 74, wherein the autophagy activator increases expression of LC3-II.
76. The method of any of embodiments 51-55, 74, or 75, wherein the autophagy activator increases the amount of LC3-II.
77. The method of any of embodiments 51-55, wherein the autophagy activator increases expression of Rab7, LAMP-2, Atg7, ULK1, ULK2, Atg5, Beclin, or c-Jun.
78. The method of any of embodiments 1-50, wherein the autophagy modulator blocks fusion of autophagosomes to lysosomes.
79. The method of embodiment 78, wherein the autophagy modulator increases the number of autophagic vesicles in the cell relative to that expected at a basal level of autophagy.
80. The method of embodiment 78, wherein the autophagy activator increases the number of autophagosomes in the cell relative to that expected at a basal level of autophagy.
81. The method of any of embodiments 78-80, wherein the autophagy modulator is Vinblastine.
82. The method of any of embodiments 78-80, wherein the autophagy modulator is Bafilomycin A1.
83. The method of any of embodiments 78-80, wherein the autophagy modulator is a COPB2 inhibitor.
84. The method of any of embodiments 78-80, wherein the autophagy modulator is capable of inhibiting COPB2.
85. The method of embodiment 83 or 84, wherein the autophagy modulator is an additional antisense compound comprising an oligonucleotide having a nucleobase sequence that is complementary to a COPB2 transcript.
86. The method of embodiment 85, wherein the additional antisense compound is an RNAi compound.
87. The method of embodiment 86, wherein the RNAi compound is an siRNA.
88. The method of embodiment 85, wherein the additional antisense compound is single-stranded.
89. The method of any of embodiments 85-88, wherein the additional antisense compound comprises a conjugate group.
90. The method of embodiment 89, wherein the additional antisense compound consists of an antisense oligonucleotide and a conjugate group.
91. The method of embodiment 85 or 88, wherein the additional antisense compound consists of an antisense oligonucleotide.
92. The method of any of embodiments 1-77, comprising contacting the cell with an additional autophagy modulator.
93. The method of embodiment 92, wherein the additional autophagy modulator blocks fusion of autophagosomes to lysosomes.
94. The method of embodiment 93, wherein the additional autophagy modulator is a COPB2 inhibitor.
95. The method of embodiment 93, wherein the additional autophagy modulator is capable of inhibiting COPB2.
96. The method of embodiment 94 or 95, wherein the additional autophagy modulator is an additional antisense compound comprising an oligonucleotide having a nucleobase sequence that is complementary to a COPB2 transcript.
97. The method of embodiment 96, wherein the second antisense compound is an RNAi compound.
98. The method of embodiment 97, wherein the RNAi compound is an siRNA.
99. The method of embodiment 96, wherein the second antisense compound is single-stranded.
100. The method of any of embodiments 96-99, wherein the second antisense compound comprises a conjugate group.
101. The method of embodiment 100, wherein the second antisense compound consists of an antisense oligonucleotide and a conjugate group.
102. The method of embodiment 96 or 99, wherein the second antisense compound consists of an antisense oligonucleotide.
103. The method of any of embodiments 1-102, wherein the antisense compound does not comprise the autophagy modulator.
104. The method of any of embodiments 1-103, wherein the autophagy modulator increases productive uptake of the antisense compound.
105. A composition comprising an autophagy modulator and an antisense compound.
106. The composition of embodiment 105, wherein the autophagy modulator is rapamycin or a rapalog.
107. The composition of embodiment 105, wherein the autophagy modulator is AZD8055.
108. The composition of embodiment 105, wherein the autophagy modulator comprises a single stranded, modified oligonucleotide and an autophagy modulator selected from rapamycin, a rapalog, and AZD8055.

### I. Certain Oligonucleotides

Provided herein are oligonucleotides, oligomeric compounds, and compositions thereof. In certain embodiments, methods disclosed herein comprise contacting a cell with an oligonucleotide, an oligomeric compound, or a composition thereof. In certain embodiments, oligonucleotides consist of linked nucleosides. In certain embodiments, an oligonucleotide is a modified oligonucleotide. In certain embodiments, the modified oligonucleotide is a modified antisense oligonucleotide. In certain embodiments, methods disclosed herein comprise contacting a cell with an antisense compound, also referred to as anoligomeric compound, which comprises an oligonucleotide. Oligonucleotides, such as antisense oligonucleotides, may comprise unmodified nucleosides. In certain embodiments, oligonucleotides comprise modified nucleosides. In general, modified oligonucleotides comprise at least one modification relative to unmodified RNA or DNA. In certain embodiments, the modification comprises a modified nucleoside. In certain embodiments, the modified nucleoside comprises a modified sugar moiety. In certain embodiments, the modified nucleoside comprises a modified nucleobase. In certain embodiments, modified nucleosides comprise a modified sugar moiety and a modified nucleobase. In certain embodiments, the modification comprises a modified internucleoside linkage. In certain embodiments, the modification comprises a modified sugar moiety or a modified internucleoside linkage.

### A. Certain Modified Nucleosides

Modified oligoucleotides disclosed herein may comprise a modified sugar moiety, a modified nucleobase, a modified internucleoside linkage, or any combination thereof

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc., 20017, 129, 8362-8379; Elayadi et al.; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

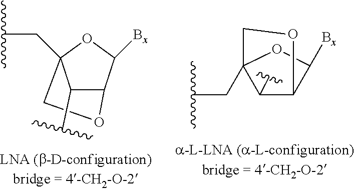

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

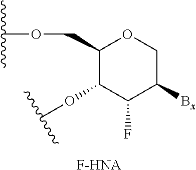

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

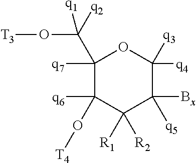

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

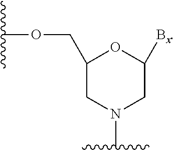

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

2. Certain Modified Nucleobases

In certain embodiments, oligonucleotides, e.g., antisense oligonucleotides, comprise one or more nucleoside comprising an unmodified nucleobase. In certain embodiments, modified oligonucleotides comprise one or more nucleoside comprising a modified nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

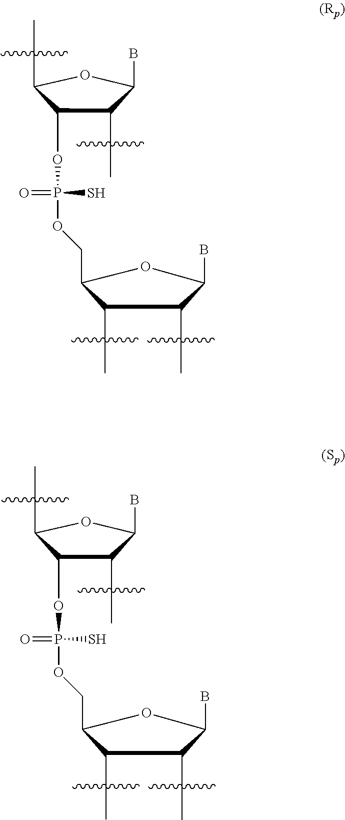

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxynucleoside. In certain embodiments, at least one nucleoside of the gap of a gapmer is a modified nucleoside.

In certain embodiments, the gapmer is a deoxy gapmer. In certain embodiments, the nucleosides on the gap side of each wing/gap junction are unmodified 2′-deoxynucleosides and the nucleosides on the wing sides of each wing/gap junction are modified nucleosides. In certain embodiments, each nucleoside of the gap is an unmodified 2′-deoxynucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

2. Certain Nucleobase Motifs

In certain embodiments, oligonucleotides, including antisense oligonucleotides, comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases are 5-methylcytosines.

In certain embodiments, modified oligonucleotides, such as modified antisense oligonucleotides, comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides, such as antisense oligonucleotides, having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides, including antisense oligonucleotides, can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### D. Certain Modified Oligonucleotides

In certain embodiments, the above modifications (sugar, nucleobase, internucleoside linkage) are incorporated into a modified oligonucleotide. In certain such embodiments, such modified oligonucleotides are antisense oligonucleotides. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides, such as antisense oligonucleotides, are further described by their nucleobase sequence. In certain embodiments, oligonucleotides have a nucleobase sequence that is complementary to a target oligonucleotide or a target nucleic acid. In certain such embodiments, a region of an oligonucleotide has a nucleobase sequence that is complementary to a target oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### II. Certain Oligomeric Compounds

Disclosed herein, in some aspects, are oligomeric compounds, which consist of an oligonucleotide (e.g., a modified, unmodified, and/or antisense oligonucleotide) and optionally one or more conjugate groups and/or terminal groups. Further disclosed herein are compositions comprising an oligomeric compound and a pharmaceutically acceptable carrier or diluent. Also disclosed herein are methods comprising contacting a cell with an oligomeric compound or a composition thereof. In certain embodiments, an oligomeric compound is also an antisense compound. In certain embodiments, an oligomeric compound is a component of an antisense compound. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain compounds comprising oligonucleotides, such as oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, compounds of the invention are single-stranded. In certain embodiments, oligomeric compounds are paired with an additional oligonucleotide or oligomeric compound to form a duplex, which is double-stranded.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid.

In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### V. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is a mRNA. In certain such embodiments, the target region is entirely within an exon. In certain embodiments, the target region spans an exon/exon junction. In certain embodiments, antisense compounds are at least partially complementary to more than one target nucleic acid.

### A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### VI. Certain Pharmaceutical Compositions

In certain embodiments, described herein are pharmaceutical compositions comprising one or more oligomeric compounds, and optionally an autophagy modulator. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### VII. Certain Combinations and Combination Therapies

In certain embodiments, methods provided herein comprise inducing autophagy in a cell and administering or contacting the cell with an antisense compound. In certain embodiments, methods provided herein comprise enhancing or increasing autophagy in a cell and administering or contacting the cell with an antisense compound.

In certain embodiments, methods disclosed herein comprise contacting a cell with an antisense oligonucleotide capable of hybridizing to a target nucleic acid in the cell and activating autophagy in the cell, thereby modifying an amount of the target nucleic acid in the cell.

In certain embodiments, methods disclosed herein comprise reducing an amount of a target nucleic acid in a cell. In certain embodiments, activating autophagy and contacting the cell with the antisense oligonucleotide reduces the amount of the target nucleic acid in the cell to a greater extent than in the absence of activating autophagy. In certain embodiments, methods comprise reducing expression of the target nucleic acid. In certain embodiments, activating autophagy and contacting the cell with the antisense oligonucleotide reduces the expression of the target nucleic acid in the cell to a greater extent than in the absence of activating autophagy.

In certain embodiments, methods disclosed herein comprise increasing an amount of a target nucleic acid in a cell. In certain embodiments, activating autophagy and contacting the cell with the antisense oligonucleotide increases the amount of the target nucleic acid in the cell to a greater extent than in the absence of activating autophagy. In certain embodiments, methods comprise increasing expression of the target nucleic acid. In certain embodiments, activating autophagy and contacting the cell with the antisense oligonucleotide increases the expression of the target nucleic acid in the cell to a greater extent than in the absence of activating autophagy.

In certain embodiments, methods comprise modifying splicing of a target nucleic acid. In certain embodiments, activating autophagy and contacting the cell with the antisense oligonucleotide increases modifies splicing of the target nucleic acid in the cell to a greater extent than in the absence of activating autophagy.

### A. Fasting Stimulates Autophagy

In certain embodiments, methods comprise fasting a subject and administering an antisense compound, or a composition thereof, to the subject. In general, the term, ‘fasting,’ as used herein, refers to caloric deprivation. A subject that is fasting may still consume water and non-caloric fluids. In certain embodiments, inducing, enhancing or increasing autophagy comprises starving a cell in vitro. In certain embodiments, methods comprise fasting a subject before administering the antisense compound to the subject. In certain embodiments, methods comprise fasting the subject for at least 16 hours before administering the antisense compound. In certain embodiments, methods comprise fasting a subject after administering an antisense compound to the subject. In certain embodiments, inducing, enhancing or increasing autophagy comprises starving a cell in vitro. In certain embodiments, starving the cell comprises culturing the cell in a starvation media.

### B. Ketogenic Diet Stimulates Autophagy

In certain embodiments, inducing, enhancing or increasing autophagy in a cell comprises restricting and/or increasing nutrients, thereby stimulating autophagy in the cell. In certain embodiments, inducing, enhancing or increasing autophagy comprises exposing the cell to a ratio of nutrients that stimulates autophagy in the cell. By way of non-limiting example, the cell may be present in a subject and the subject may be fed a ketogenic diet, thereby inducing autophagy in cells of the subject. A ketogenic diet may be defined by percentages of caloric intake obtained from fat, protein and carbohydrates. In certain embodiments, at least 90% of caloric intake is from fat. In certain embodiments, at least 85% of caloric intake is from fat. In certain embodiments, at least 80% of caloric intake is from fat. In certain embodiments, at least 75% of caloric intake is from fat.

In certain embodiments, methods comprise exposing a subject to a ketogenic diet and administering an antisense compound to the subject. In certain embodiments, methods comprise exposing a subject to a ketogenic diet before administering an antisense compound to the subject. In certain embodiments, methods comprise exposing a subject to a ketogenic diet after administering an antisense compound to the subject. In certain embodiments, methods comprise administering an antisense compound to a subject while the subject is on a ketogenic diet. In certain embodiments, methods comprise administering the antisense compound to the subject, wherein the subject has followed a ketogenic diet for at least one day, at least two days, at least three days, or at least five days before the administering. In certain embodiments, methods comprise administering the antisense compound to the subject, wherein the subject has followed a ketogenic diet for at least one week before the administering. In certain embodiments, methods comprise administering the antisense compound to the subject, wherein the subject has followed a ketogenic diet for at least two weeks, at least three weeks, or at least five weeks before the administering. In certain embodiments, the subject follows a ketogenic diet for at least one day, at least two days, at least three days, or at least five days after the administering. In certain embodiments, the subject follows a ketogenic diet for at least one week after the administering. In certain embodiments, the subject follows a ketogenic diet for at least two weeks, at least three weeks, or at least four weeks after the administering.

### C. Autophagy Modulators

In certain embodiments, methods provided herein comprise contacting a cell with an antisense compound and an autophagy modulator, wherein the autophagy modulator increases the activity of the antisense compound in the cell relative to the activity of the antisense compound in the cell in the absence of the autophagy modulator. In certain embodiments, the cell is in vivo and methods comprise administering the autophagy modulator and antisense compound to the subject. In certain embodiments, methods comprise adeministering an antisense compound and one or more autophagy modulators in combination. In certain embodiments, methods comprise administering an antisense compound and an autophagy modulator simultaneously. In certain embodiments, methods comprise administering an antisense compound and an autophagy modulator separately. In certain embodiments, methods comprise administering an antisense compound and an autophagy modulator sequentially. In certain embodiments, the antisense compound and the autophagy modulator are formulated as a fixed dose combination product. In other embodiments, they are provided to the patient as separate units which can then either be taken simultaneously or serially (sequentially).

In certain embodiments, administration of the autophagy modulator and antisense compound permits use of a lower dosage of the antisense compound than would be required to achieve a therapeutic or prophylactic effect if the antisense compound were administered alone. In certain embodiments, co-administration of the antisense compound and the autophagy modulator permits use of a lower dosage of the antisense compound to achieve a therapeutic result, wherein the lower dosage is lower than a comparative dosage of the antisense compound necessary to achieve the therapeutic result when the antisense compound is used without the autophagy modulator. In certain embodiments, methods comprise inducing or enhancing autophagy via fasting or nutrient restriction. In certain embodiments, inducing or enhancing autophagy permits use of a lower dose of the antisense compound to achieve a therapeutic result relative to a comparative dose of the antisense compound when the compound is administered in the absence of inducing or enhancing autophagy.

In certain embodiments, methods comprise co-administering an antisense compound comprising or consisting of an antisense oligonucleotide with one or more autophagy modulators. In certain such embodiments, the antisense compound and one or more autophagy modulators are administered simultaneously. In certain embodiments, an antisense compound comprising or consisting of an antisense oligonucleotide and one or more autophagy modulators are administered to a subject sequentially. In certain such embodiments, the antisense compound and one or more autophagy modulators are administered at different times. In certain embodiments, the antisense compound is administered before the one or more autophagy modulators. In certain embodiments, the antisense compound is administered after the one or more autophagy modulators.

In certain embodiments, methods comprise administering the antisense compound and one or more autophagy modulators together in a single formulation. In certain embodiments, compositions disclosed herein comprise an autophagy modulator and an antisense oligonucleotide in a single formulation. In certain embodiments, methods comprise administering the antisense compound and one or more autophagy modulators, wherein the antisense compound and one or more autophagy modulators are prepared as separate formulations.

In certain embodiments, compositions and methods disclosed herein comprise an autophagy modulator, or a use thereof. In certain embodiments, the autophagy modulator is a small molecule. In certain embodiments, the autophagy modulator is an autophagy activator. In certain embodiments, the autophagy activator induces autophagosome formation. In certain embodiments, the autophagy activator induces autophagosome nucleation. In certain embodiments, the autophagy activator induces autophagosome elongation. In certain embodiments, the autophagy activator increases the number of autophagosomes in the cell. In certain embodiments, the autophagy activator increases expression of an autophagy pathway component. In certain embodiments, the autophagy activator increases autophagosome formation relative to that expected at a basal level of autophagy. In certain embodiments, the autophagy activator increases autophagosome nucleation relative to a basal level of autophagy in the absence of the autophagy activator. In certain embodiments, the autophagy activator increases autophagosome elongation relative to a basal level of autophagy in the absence of the autophagy activator. In certain embodiments, the autophagy activator increases the number of autophagic vesicles in the cell relative to a basal level of autophagy in the absence of the autophagy activator. In certain embodiments, the autophagy activator increases the number of autophagosomes in the cell relative to a basal level of autophagy in the absence of the autophagy activator.

In certain embodiments, compositions and methods disclosed herein comprise an autophagy modulator, or a use thereof, wherein the autophagy activator is an mTor inhibitor. In certain embodiments, the mTor inhibitor is capable of inhibiting mTor activity in the cell. In certain embodiments, mTor activity comprises kinase activity, protein binding activity, or a comabination thereof. In certain embodiments, the mTor inhibitor comprises rapamycin. In certain embodiments, the mTor inhibitor is rapamycin. In certain embodiments, the mTor inhibitor comprises a rapalog. In certain embodiments, the rapalog is temsirolimus. In certain embodiments, the rapalog is everolimus. In certain embodiments, the rapalog is ridaforolimus. In certain embodiments, the autophagy activator is an ATP-competitive mTor inhibitor. Non-limiting examples of ATP-competitive mTor inhibitors are OSI-027, AZD8055, AZD2014, and INK128. In certain embodiments, the autophagy activator is AZD8055. In certain embodiments, the autophagy activator selectively inhibits mTor over other kinases. In certain embodiments, the autophagy activator selectively inhibits mTor over PI3 kinase. In certain embodiments, the autophagy activator inhibits mTor and at least one other kinase. In certain embodiments, the autophagy activator is not a PI3 kinase inhibitor. In certain embodiments, the autophagy activator is PI-103. In certain embodiments, the autophagy activator is PP242. In certain embodiments, the autophagy activator increases expression of LC3. In certain embodiments, the autophagy activator increases expression of LC3-II. In certain embodiments, the autophagy activator increases the amount of LC3-II. In certain embodiments, the autophagy activator increases expression of Rab7, LAMP-2, Atg7, ULK1, ULK2, Atg5, Beclin, or c-Jun.

In certain embodiments, compositions and methods disclosed herein comprise an autophagy modulator, or a use thereof, wherein the autophagy activator blocks fusion of autophagosomes to lysosomes. In certain embodiments, the autophagy modulator increases the number of autophagic vesicles in the cell relative to that expected at a basal level of autophagy. In certain embodiments, the autophagy activator increases the number of autophagosomes in the cell relative to that expected at a basal level of autophagy. In certain embodiments, the autophagy modulator is Vinblastine. In certain embodiments, the autophagy modulator is Bafilomycin A1. In certain embodiments, the autophagy modulator is a COPB2 inhibitor. In certain embodiments, the autophagy modulator is capable of inhibiting COPB2.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and other publications recited in the present application is incorporated herein by reference in its entirety.

Although the sequence listing accompanying this filing identifies each sequence as either “RNA” or

“DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., antisense oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their racemic and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

The following examples demonstrate autophagy enhancement of antisense activity in a wide variety of cell types (mouse and human) with a variety of antisense compounds and a variety of autophagy modulating compounds to demonstrate that the effects of autophagy modulation on antisense activity are not limited to any one of these parameters. A wide variety of autophagy activating compounds enhanced antisense oligonucleotide activity in primary cells, embryonic fibroblasts and cell lines from rodents and humans. Cell types in which autophagy modulators enhanced antisense oligonucleotide activity included cells originating from liver, skin, neuronal tissue, cervical epithelium, connective tissue, and lung, as well as mouse embryonic fibroblasts, and human fibroblasts.

The following examples also demonstrate that the antisense activity enhancing effects of autophagy activating compounds are observed in vivo, and that conditions that activate autophagy, such as fasting and ketogenic diet enhance antisense activity in mice.

Further, the following examples demonstrate that antisense oligonucleotide can be chemically modified in various ways without abolishing the antisense activity enhancing effects of autophagy activation. Several antisense oligonucleotides were 5-10-5 MOE gapmers, while others were 3-10-3 cEt gapmers, and still others comprised a GalNAc moiety.

### Example 1: Effect of Modulation of Autophagy on Antisense Activity In Vitro, Single Dose

The following experiment was performed to determine if compounds known to modulate autophagy affect antisense activity.

### Cell Treatment

MHT cells (a mouse hepatocellular carcinoma cell line) were plated at 20,000 cells/well and treated with DMSO (control) or an autophagy modulator for 24 hours at the concentrations indicated in Table 2 below. 3-MA, BafA1, AZD8055, and leupeptin are autophagy activators. Chloroquine is an autophagy inhibitor. Fourteen hours after the addition of autophagy modulators, 10 μM of a modified control oligonucleotide or of a modified antisense oligonucleotide was added by free uptake for ten hours. The modified oligonucleotides, shown in Table 1 below, are 3-10-3 cEt gapmers in which every internucleoside linkage is a phosphorothioate internucleoside linkage. The three 5′-terminal nucleosides and the three 3′-terminal nucleosides of each oligonucleotide each comprise a cEt modified sugar moiety. The remaining ten nucleosides of each oligonucleotide each comprise an unmodified, 2′-deoxy sugar moiety. All of the cytosines had a methyl at the 5 position.

TABLE 1
Modified antisense oligonucleotides

| | | Target to which | |
|---|---|---|---|
| Compound | | it is 100% | SEQ |
| No. | Sequence | complementary | ID NO |
| 549148 | GGCTACTACGCCGTCA | none (control) | 1 |
| 556089 | GCATTCTAATAGCAGC | Malat-1 | 2 |

TABLE 2
Autophagy Modulators

| | Concen- | |
|---|---|---|
| | tration | |
| Compound | used | |
| 3-methyladenine | 5 | mM |
| (3-MA) | | |
| Bafilomycin | 80 | nM |
| A1 (BafA1) | | |
| AZD8055 | 500 | nM |
| Leupeptin | 40 | μM |
| Chloroquine | 25 | μM |

### RNA Analysis

Using the primer probe sets described in Table 3 below, RT-qPCR analysis was performed to detect RNA levels of Malat-1, shown in Table 4 and markers of autophagy, (Rab7, LAMP2, Atg7, ULK1, ULK2, Atg5, Beclin, and c-Jun), shown in Table 5. Results were normalized to cyclophilin A levels. To control for global effects of autophagy modulators on gene expression, Malat-1 RNA levels after treatment with an antisense oligonucleotide and an autophagy modulator were normalized to Malat-1 RNA levels in cells treated with control Compound No. 549148 and the same autophagy modulator.

The results in Table 4 demonstrate that induction of autophagy by 3-MA, BafA1, AZD8055 and leupeptin significantly enhanced antisense activity. An inhibitor of autophagy (chloroquine) reduced antisense activity relative to cells treated with DMSO.

TABLE 3
Primer Probe sets

| | | | | SEQ ID |
|---|---|---|---|---|
| Transcript | PP Set Name | Primer | Sequence | NO: |
| Malat1 | 15877 | Forward | TGGGTTAGAGAAGGCGTGTACTG | 3 |
| | | Reverse | TCAGCGGCAACTGGGAAA | 4 |
| | | Probe | CGTTGGCACGACACCTTCAGGGA | 5 |
| | | | CT | |
| Rab7 | 39907 | Forward | TTCAGTAACCAGTACAAAGCCA | 6 |
| | | Reverse | GTAGAAGGCCACACCAAGAG | 7 |
| | | Probe | CTGTCGTCCACCATCACCTCCTT | 8 |
| | | | G | |
| LAMP-2 | Mm00495267_m1 | ThermoFisher TaqMan Assay | | |
| Atg7 | 36629 | Forward | CCAGATTGTCCTAAAGCAGTTG | 9 |
| | | Reverse | ACCAATCTCCAACACATCAGT | 10 |
| | | Probe | TGGTGAACCTCAGTGAATGTATG | 11 |
| | | | GACC | |
| ULK1 | 33586 | Forward | CCAAGTCCCAAACACTGCT | 12 |
| | | Reverse | CCAGGTAGACAGAATTAGCCAT | 13 |
| | | Probe | CTGGAAGTCATACAGCGCCACGA | 14 |
| | | | T | |
| ULK2 | Mm03048846_ml | ThermoFisher TaqMan Assay | | |
| Atg5 | 39522 | Forward | GACGAATTCCAACTTGCTTTACT | 15 |
| | | | C | |
| | | Reverse | GTCAGTTACCAACGTCAAATAGC | 16 |
| | | Probe | TGGTTCTGCTTCTCTTTCAGTTA | 17 |
| | | | TCTCATCCT | |
| Beclin 1 | 33587 | Forward | GTCTTCAATGCCACCTTCCA | 18 |
| | | Reverse | GCATTGATTTCATTCCACTCCA | 19 |
| | | Probe | TTGTGCCAAACTGTCCGCTGTG | 20 |
| c-Jun | 387 | Forward | GAAGTGACGGACCGTTCTATGAC | 21 |
| | | Reverse | GGAGGAACGAGGCGTTGA | 22 |
| | | Probe | TGGAAACGACCTTCTACGACGAT | 23 |
| | | | GCC | |

TABLE 4
Malat-1 RNA levels with cET gapmers in MHTs

| | Malat-1 level |
|---|---|
| | (% relative to |
| | 549148 + auto- |
| Cell treatment | phagy modulator) |
| 556089 + DMSO | 16 |
| 556089 + 3-MA | 0 |
| | (none detected) |
| 556089 + BafA1 | 4 |
| 556089 + AZD8055 | 0 |
| | (none detected) |
| 556089 + Leupeptin | 10 |
| 556089 + Chloroquine | 54 |

TABLE 5
mRNA levels for autophagy markers

| | mRNA levels (% PBS control) | | | | | | | |
|---|---|---|---|---|---|---|---|---|
| Cell treatment | Rab7 | LAMP-2 | Atg7 | ULK1 | ULK2 | Atg5 | Beclin | c-Jun |
| PBS | 100 | 100 | 100 | 100 | 100 | 100 | 100 | 100 |
| 549148 + DMSO | 96 | 105 | 107 | 108 | 96 | 102 | 107 | 92 |
| 549148 + 3-MA | 133 | 185 | 175 | 147 | 153 | 149 | 177 | 122 |
| 549148 + BafA 1 | 144 | 171 | 114 | 98 | 111 | 102 | 126 | 184 |
| 549148 + AZD9055 | 119 | 154 | 160 | 128 | 137 | 94 | 132 | 102 |
| 549148 + Leupeptin | 110 | 154 | 103 | 101 | 99 | 102 | 91 | 83 |
| 549148 + Chloroquine | 108 | 105 | 102 | 83 | 96 | 86 | 87 | 70 |
| 556089 + DMSO | 91 | 96 | 108 | 98 | 98 | 97 | 98 | 97 |
| 556089 + 3-MA | 131 | 188 | 173 | 159 | 154 | 145 | 154 | 125 |
| 556089 + BafA 1 | 141 | 158 | 111 | 89 | 108 | 106 | 117 | 182 |
| 556089 + AZD9055 | 116 | 145 | 165 | 122 | 133 | 94 | 132 | 110 |
| 556089 + Leupeptin | 115 | 124 | 114 | 96 | 99 | 100 | 101 | 85 |
| 556089 + Chloroquine | 116 | 119 | 119 | 87 | 94 | 89 | 100 | 69 |

### Example 2: Effect of Modulation of Autophagy on Antisense Activity In Vitro, Multiple Doses of Antisense Oligonucleotides

The following experiment was performed to determine if the effects of autophagy modulators observed in Example 1 are observed as antisense oligonucleotide dose is varied. Moreover, in an effort to determine if the effects of autophagy modulators observed in Example 1 are preserved in the presence of a different antisense oligonucleotide chemical modification pattern, antisense oligonucleotides in the following example have a different pattern of chemical modifications (5-10-5 MOE gapmers) than those used in Example 1 (3-10-3 cEt gapmers).

### Cell Treatment

MHT cells were plated at 20,000 cells/well and treated with DMSO (control) or an autophagy modulator for 24 hours, at the concentrations indicated Table 7 below. Fourteen hours after the addition of autophagy modulator, control or antisense oligonucleotides (see Table 6) were added at 1 μM, 2.5 μM, 5 μM, or 10 μM by free uptake for ten hours. The modified oligonucleotides, shown in Table 6 below, are 5-10-5 MOE gapmers in which every internucleoside linkage is a phosphorothioate internucleoside linkage. The five 5′-terminal nucleosides and the five 3′-terminal nucleosides of each oligonucleotide each comprise a 2′-MOE modified sugar moiety. The remaining ten nucleosides of each oligonucleotide each comprise an unmodified, 2′-deoxy sugar moiety. All of the cytosines had a methyl at the 5 position.

TABLE 6
Modified oligonucleotides

| | | Target to which | SEQ |
|---|---|---|---|
| Compound | | it is 100% | ID |
| No. | Sequence | complementary | NO |
| 141923 | CCTTCCCTGAAGGTTCCTCC | none (control) | 24 |
| 399462 | GGGTCAGCTGCCAATGCTAG | Malat-1 | 25 |

TABLE 7
Autophagy Modulators

| Compound | Concentration used | |
|---|---|---|
| Rapamycin | 500 | nM |
| VPS34-IN2 | 5 | μM |
| Vinblastine | 50 | μM |
| wortmannin | 1 | μM |
| E64D | 10 | μM |

### RNA Analysis

RT-qPCR analysis was performed to detect RNA levels of Malat-1 using primer probe set 15877 (described in Example 1). In order to control for global effects of autophagy modulators on gene expression, Malat-1 RNA levels after treatment with an antisense oligonucleotide and an autophagy modulator were normalized to Malat-1 RNA levels in cells treated with control Compound No. 141923 and the same autophagy modulator. Each table below represents results from an individual assay.

Tables 8-12 show the results of five experiments performed separately. All results demonstrate that induction of autophagy by 3-MA, BafA1, AZD8055, rapamycin, VPS34-IN2, vinblastine, and leupeptin significantly enhanced modified oligonucleotide activity of MOE gapmers in MHT cells relative to cells treated with control DMSO. In contrast, inhibitors of autophagy (wortmannin, E64D, and chloroquine) reduced activity of modified oligonucleotide relative to cells treated with control DMSO. These results were observed regardless of antisense oligonucleotide dose and specific autophagy modulator. Together the results of Example 1 and Example 2 suggest that autophagy modulators enhance the activity of antisense oligonucleotides regardless of antisense oligonucleotide chemical modifications.

TABLE 8
Malat-1 RNA levels with MOE gapmer in MHTs

| | Malat-1 level (% relative to | | |
|---|---|---|---|
| | 141923 + autophagy modulator) | | |
| | 2.5 μM | 5 μM | 10 μM |
| Cell treatment | 399462 | 399462 | 399462 |
| 399462 + DMSO | 55 | 62 | 41 |
| 399462 + 3-MA | 45 | 50 | 37 |
| 399462 + BafA1 | 34 | 29 | 14 |
| 399462 + AZD8055 | 43 | 35 | 11 |
| 399462 + Leupeptin | 58 | 73 | 34 |
| 399462 + Chloroquine | 91 | 104 | 92 |

TABLE 9
Malat-1 RNA levels with MOE gapmer in MHTs

| | Malat-1 level (% relative to | | |
|---|---|---|---|
| | 141923 + autophagy modulator) | | |
| | 2.5 μM | 5 μM | 10 μM |
| Cell treatment | 399462 | 399462 | 399462 |
| 399462 + DMSO | 118 | 92 | 56 |
| 399462 + AZD8055 | 52 | 48 | 43 |
| 399462 + Rapamycin | 87 | 57 | 60 |
| 399462 + Chloroquine | 144 | 110 | 83 |

TABLE 10
Malat-1 RNA levels with MOE gapmer in MHTs

| | Malat-1 level (% relative to | | |
|---|---|---|---|
| | 141923 + autophagy modulator) | | |
| | 2.5 μM | 5 μM | 10 μM |
| Cell treatment | 399462 | 399462 | 399462 |
| 399462 + DMSO | 84 | 62 | 51 |
| 399462 + AZD8055 | 0 | 0 | 0 |
| | (none detected) | (none detected) | (none detected) |
| 399462 + Chloroquine | 96 | 98 | 98 |
| 399462 + VPS34-1N | 84 | 81 | 75 |

TABLE 11
Malat-1 RNA levels with MOE gapmer in MHTs

| | Malat-1 level (% relative to |
|---|---|
| | 141923 + autophagy modulator) |
| Cell treatment | 2.5 μM 399462 |
| 399462 + DMSO | 62 |
| 399462 + AZD8055 | 31 |
| 399462 + Chloroquine | 111 |
| 399462 + vinblastine | 39 |

TABLE 12
Malat-1 RNA levels with MOE gapmer in MHTs

| | Malat-1 level (% relative to | | |
|---|---|---|---|
| | 141923 + autophagy modulator) | | |
| | 1 μM | 2.5 μM | 5 μM |
| Cell treatment | 399462 | 399462 | 399462 |
| 399462 + DMSO | 57 | 58 | 47 |
| 399462 + wortmannin | 74 | 64 | 54 |
| 399462 + E64D | 62 | 75 | 58 |

### Example 3: Effect of Modulation of Autophagy on Antisense Activity In Vitro in MHT Cells (Mouse Hepatocellular Carcinoma Cell Model), Multiple Dose

To determine whether the effects of autophagy on antisense activity observed in Examples 1 and 2 would be preserved with an antisense oligonucleotide targeting a different nucleic acid, experiments similar to those in Examples 1 and 2 were repeated with an antisense oligonucleotide targeting SRB1 (instead of Malat1).

### Cell Treatment

MHT cells were plated at 20,000 cells/well and treated with DMSO (control) or an autophagy modulator for 24 hours, at the concentrations indicated in Example 1. Fourteen hours after the addition of an autophagy modulator, a modified control oligonucleotide or modified antisense oligonucleotide was added at 2.5 μM, 5 μM, or 10 μM by free uptake for ten hours, as indicated in the tables below. The modified oligonucleotides, shown in Table 13 below, are 5-10-5 MOE gapmers in which every internucleoside linkage is a phosphorothioate internucleoside linkage. The five 5′-terminal nucleosides and the five 3′-terminal nucleosides of each oligonucleotide each comprise a 2′-MOE modified sugar moiety. The remaining ten nucleosides of each oligonucleotide each comprise an unmodified, 2′-deoxy sugar moiety. All the cytosines had a methyl at the 5 position.

TABLE 13
Modified oligonucleotides

| | | Target to which | SEQ |
|---|---|---|---|
| Compound | | it is 100% | ID |
| No. | Sequence | complementary | NO |
| 141923 | CCTTCCCTGAAGGTTCCTCC | none (control) | 24 |
| 353382 | GCTTCAGTCATGACTTCCTT | SRB1 | 26 |

mRNA Analysis

RT-qPCR analysis was performed to detect mRNA levels of SRB1 using primer probe set 15299 (forward sequence: TGACAACGACACCGTGTCCT, SEQ ID NO:27; reverse sequence: ATGCGACTTGTCAGGCTGG, SEQ ID NO:28; probe sequence: CGTGGAGAACCGCAGCCTCCATT, SEQ ID NO:29). Results were normalized to cyclophilin A levels. To control for global effects of autophagy modulators on gene expression, SRB1 mRNA levels after treatment with an antisense oligonucleotide and an autophagy modulator were normalized to SRB1 mRNA levels in cells treated with control Compound No. 141923 and the same autophagy modulator.

Results presented in Table 14 demonstrate that induction of autophagy significantly enhanced activity of modified oligonucleotides (MOE gapmers) targeting SRB1 in MHT cells. In contrast, inhibition of autophagy reduced antisense activity.

TABLE 14
SRB1 mRNA levels in MHT cells with MOE gapmer

| | SRB1 level (% relative to | | |
|---|---|---|---|
| | 141923 + autophagy modulator) | | |
| | 2.5 μM | 5 μM | 10 μM |
| Cell treatment | 353382 | 353382 | 353382 |
| 353382 + DMSO | 50 | 49 | 45 |
| 353382 + AZD8055 | 32 | 27 | 27 |
| 353382 + Chloroquine | 98 | 97 | 93 |

### Example 4: Effect of Modulation of Autophagy on Antisense Activity in HeLa Cells (Human Cervical Cancer Cell Line), Multiple Dose

Examples 1-3 demonstrate that activating autophagy in a liver cell line enhances antisense activity against multiple targets regardless of antisense oligonucleotide chemical modifications. To query whether these results were cell-type specific or species specific, similar experiments were repeated in a human epithelial cell line derived from a cervical cancer cells (HeLa).

### Cell Treatment

HeLa cells were plated at 10,000 cells/well and treated with DMSO (control) or an autophagy modulator for 24 hours, at the concentrations indicated in Example 1 or in the tables below. Fourteen hours after the addition of autophagy modulator, control or antisense oligonucleotides were added at 2.5 μM, 5 μM, or 10 μM by free uptake for ten hours, as indicated in the tables below. The modified oligonucleotides, shown in Table 15 below, are 5-10-5 MOE gapmers in which every internucleoside linkage is a phosphorothioate internucleoside linkage. The five 5′-terminal nucleosides and the five 3′-terminal nucleosides of each oligonucleotide each comprise a 2′-MOE modified sugar moiety. The remaining ten nucleosides of each oligonucleotide each comprise an unmodified, 2′-deoxy sugar moiety. All of the cytosines had a methyl at the 5 position.

TABLE 15
Modified oligonucleotides

| | | Target to which | SEQ |
|---|---|---|---|
| Compound | | it is 100% | ID |
| No. | Sequence | complementary | NO |
| 395254 | GGCATATGCAGATAATGTTC | MALAT1 | 31 |

### RNA Analysis

RT-qPCR analysis was performed to detect RNA levels of Malat-1 using primer probe set RTS2736 (forward sequence AAAGCAAGGTCTCCCCACAAG, designated herein as SEQ ID NO: 32; reverse sequence TGAAGGGTCTGTGCTAGATCAAAA, designated herein as SEQ ID NO: 33; probe sequence TGCCACATCGCCACCCCGT, designated herein as SEQ ID NO: 34). Results were normalized to cyclophilin A levels, measured using human cyclophilin A primer probe set HTS3936 (forward sequence GCCATGGAGCGCTTTGG, designated herein as SEQ ID NO: 35; reverse sequence TCCACAGTCAGCAATGGTGATC, designated herein as SEQ ID NO: 36; probe sequence TCCAGGAATGGCAAGACCAGCAAGA, designated herein as SEQ ID NO: 37). In order to control for global effects of autophagy modulators on gene expression, Malat-1 RNA levels after treatment with an antisense oligonucleotide and an autophagy modulator were normalized to Malat-1 RNA levels in cells treated with control Compound No. 141923.

As shown in Table 16, induction of autophagy significantly enhanced activity of modified oligonucleotides (MOE gapmers) targeting MALAT1 in HeLa cells. In contrast, inhibition of autophagy reduced antisense activity.

TABLE 16
Malat-1 RNA levels in HeLA cells with MOE gapmer

| | Malat-1 level (% relative | | |
|---|---|---|---|
| | to 141923 + autophagy modulator) | | |
| | 2.5 μM | 5 μM | 10 μM |
| Cell treatment | 399462 | 399462 | 399462 |
| 395254 + DMSO | 82 | 78 | 70 |
| 395254 + AZD8055 | 55 | 54 | 57 |
| 395254 + Chloroquine | 77 | 79 | 80 |

### Example 5: Effect of Modulation of Autophagy on Antisense Activity In Vitro in Epidermoid Carcinoma

Effects of autophagy modulators on antisense activity were also tested in a human cell line derived from an epidermoid carcinoma in the skin (epidermis), A431 cells.

### Cell Treatment

A431 cells were plated at 16,300 cells/well and treated with DMSO (control) or an autophagy modulator for 24 hours, at the concentrations indicated in Example 1. Fourteen hours after the addition of an autophagy modulator, a modified control oligonucleotide (Compound No. 141923 described in examples above) or modified antisense oligonucleotide (Compound No. 395254 described in examples above) was added by free uptake at 0.01 μM, 0.1 μM, 1 μM, 2.5 μM, or 5 μM for ten hours.

### RNA Analysis

RT-qPCR analysis was performed to detect RNA levels of Malat-1 using primer probe set RTS2736. Results were normalized to cyclophilin A levels (measured using HTS3936). In order to control for global effects of autophagy modulators on gene expression, Malat-1 RNA levels after treatment with an antisense oligonucleotide and an autophagy modulator were normalized to Malat-1 RNA levels in cells treated with control Compound No. 141923. Each table below represents results from an individual assay.

As demonstrated in three separate experiments, (results shown in Tables 17-19, respectively), induction of autophagy by BafA1, AZD8055, rapamycin, VPS34-IN2, and leupeptin significantly enhanced modified oligonucleotide activity of MOE gapmers in A431 cells. An inhibitor of autophagy (chloroquine) reduced activity of the modified oligonucleotide.

TABLE 17
Malat-1 RNA levels

| | Malat-1 level (% relative to | | |
|---|---|---|---|
| | 141923 + autophagy modulator) | | |
| Cell treatment | 1 μM 399462 | 2.5 μM 399462 | 5 μM 399462 |
| 395254 + DMSO | 15.6 | 11.4 | 10.2 |
| 395254 + AZD8055 | 7.0 | 5.8 | 5.0 |
| 395254 + Chloroquine | 38.4 | 27.7 | 24.8 |
| 395254 + BafAl | 12.9 | 9.1 | 8.7 |

TABLE 18
Malat-1 RNA levels

| | Malat-1 level (% relative to | | |
|---|---|---|---|
| | 141923 + autophagy modulator) | | |
| Cell treatment | 0.01 μM 399462 | 0.1 μM 399462 | 1 μM 399462 |
| 395254 + DMSO | 41.8 | 24.0 | 11.2 |
| 395254 + AZD8055 | 23.8 | 12.7 | 7.5 |
| 395254 + Chloroquine | 92.5 | 76.4 | 46.8 |
| 395254 + Rapamycin | 30.6 | 15.8 | 7.8 |

TABLE 19
Malat-1 RNA levels

| | Malat-1 level (% relative to | | |
|---|---|---|---|
| | 141923 + autophagy modulator) | | |
| Cell treatment | 0.01 μM 399462 | 0.1 μM 399462 | 1 μM 399462 |
| 395254 + DMSO | 50 | 19 | 10 |
| 395254 + BafAl | 59 | 32 | 16 |
| 395254 + VPS34-IN2 | 58 | 19 | 8 |
| 395254 + Leupeptin | 44 | 26 | 11 |

### Example 6: Effect of Modulation of Autophagy Prior to Antisense Oligonucleotide Treatment

Cells were treated with autophagy modulators before treatment with antisense oligonucleotides in Examples 1-5. In contrast, cells in the following experiment were treated with autophagy modulators after treatment with antisense oligonucleotides.

### Cell Treatment

MHT cells were plated at 15,000 cells/well and treated with DMSO (control), a control oligonucleotide (Compound No. 141923), or an antisense oligonucleotide (Compound No. 353382 or 399462), described in the Examples above, by free uptake for 24 hours at 0.01 μM, 0.1 μM, 1 μM, 2.5 μM, or 5 μM. Media was then removed and exchanged for media containing DMSO, 25 μM chloroquine, 500 nM AZD8055, or 500 nM rapamycin for 24 hours prior to harvest and analysis by RT-qPCR as described in Example 1 for Malat-1 or in Example 3 for SRB1.

A significant improvement in modified oligonucleotide mediated RNA knockdown was observed for multiple targets even when treatment with modified oligonucleotide was started prior to autophagy induction, as evidenced by the results shown in Tables 20-22.

TABLE 20
SRB1 mRNA levels

| | SRB1 level (% relative to | | |
|---|---|---|---|
| | 141923 + autophagy modulator) | | |
| Cell treatment | 1 μM 353382 | 2.5 μM 353382 | 5 μM 353382 |
| 353382, then DMSO | 20.7 | 18.1 | 17.9 |
| 353382, then chloroquine | 20.5 | 19.1 | 17.3 |
| 353382, then AZD8055 | 13.4 | 12.1 | 12.3 |

TABLE 21
SRB1 mRNA levels

| | SRB1 level (% relative to | | |
|---|---|---|---|
| | 141923 + autophagy modulator) | | |
| Cell treatment | 0.01 μM 353382 | 0.1 μM 353382 | 1 μM 353382 |
| 353382, then DMSO | 24.3 | 19.5 | 16.7 |
| 353382, then chloroquine | 34.1 | 38.1 | 26.3 |
| 353382, then AZD8055 | 15.7 | 17.0 | 13.0 |
| 353382, then rapamycin | 19.4 | 21.2 | 13.4 |

TABLE 22
Malat-1 RNA levels

| | Malat-1 level (% relative to | | |
|---|---|---|---|
| | 141923 + autophagy modulator) | | |
| Cell treatment | 0.01 μM 399462 | 0.1 μM 399462 | 1 μM 399462 |
| 399462, then DMSO | 45.4 | 23.9 | 15.2 |
| 399462, then chloroquine | 51.9 | 42.6 | 14.8 |
| 399462, then AZD8055 | 27.5 | 13.8 | 4.9 |
| 399462, then rapamycin | 41.5 | 24.1 | 8.4 |

### Example 7: Kinetic Effects of Autophagy Modulation on Antisense Activity in MHT Cells

### Cell Treatment

MHT cells were plated at 20,000 cells/well and treated with DMSO (control) or AZD8055 for 24 hours. Modified oligonucleotides described above were then added by free uptake for 40 minutes, 2 hours, or 4 hours, then the cells were incubated with fresh media (containing no oligonucleotide or autophagy modulator) for 2 hours prior to cell harvest.

### RNA Analysis

RT-qPCR analysis was performed to detect RNA levels of Malat-1 using primer probe set 15877 (described in Example 1). Results (shown in Table 23) were normalized to cyclophilin A levels. In order to control for global effects of autophagy modulators on gene expression, Malat-1 RNA levels after treatment with an antisense oligonucleotide and an autophagy modulator were normalized to Malat-1 RNA levels in cells treated with control Compound No. 141923.

Autophagy mediated enhancement of modified oligonucleotide activity was observed as early as 40 minutes after treatment and increased significantly over time.

TABLE 23
Malat-1 RNA levels

| | Malat-1 level (% relative to | | |
|---|---|---|---|
| | 141923 + autophagy modulator) | | |
| | 40 minutes | 2 hours | 4 hours |
| Cell treatment | with 399462 | with 399462 | with 399462 |
| DMSO + 0.1 μM 399462 | 116 | 103 | 99 |
| AZD8055 + 0.1 μM 399462 | 101 | 83 | 49 |
| DMSO + 2 μM 399462 | 108 | 87 | 74 |
| AZD8055 + 2 μM 399462 | 80 | 69 | 44 |

### Example 8: Confocal Imaging of Autophagic Vesicles

The Premo autophagy tandem sensor RFP-GFP-LC3B (ThermoFisher, P36239) was used to visualize autophagy in MHT cells. Cells were treated with the Premo sensor and either DMSO or AZD8055 for 24 hours prior to the addition of modified antisense oligonucleotide, Compound No. 851810, at 0.1 μM or 2 μM by free uptake for 40 minutes, 2 hours, or 4 hours. Confocal images were analyzed by counting the total number of autophagic vesicles per cell and the number of autophagic vesicles that colocalized with antisense oligonucleotide. Results represent the average of 8-12 images. Results are shown in Table 24.

Compound No. 851810 comprises a 5-10-5 MOE gapmer of the sequence CTGCTAGCCTCTGGATTTGA (SEQ ID NO: 30) and an AlexaFluor647 conjugated to the 5′-end of the oligonucleotide. Every internucleoside linkage of Compound No. 851810 is a phosphorothioate internucleoside linkage. The five 5′-terminal nucleosides and the five 3′-terminal nucleosides of the compound each comprise a 2′-MOE modified sugar moiety. The remaining ten nucleosides each comprise an unmodified, 2′-deoxy sugar moiety. All the cytosines had a methyl at the 5 position. Compound No. 851810 is 100% complementary to human PTEN mRNA.

TABLE 24
Colocalization of antisense oligonucleotide with autophagic vesicles

| | Total number | | | % of autophagic vesicles | | |
|---|---|---|---|---|---|---|
| | autophagic | | | co-localized with antisense | | |
| | vesicles/cell | | | oligonucleotide | | |
| | 40 | 2 | 4 | 40 | 2 | 4 |
| Cell treatment | minutes | hours | hours | minutes | hours | hours |
| DMSO + 0.1 μM 851810 | 24 | 26 | 30 | 19 | 35 | 33 |
| AZD8055 + 0.1 μM 851810 | 146 | 153 | 191 | 30 | 47 | 68 |
| DMSO + 2 μM 851810 | 28 | 33 | 39 | 15 | 49 | 55 |
| AZD8055 + 2 μM 851810 | 129 | 167 | 115 | 43 | 73 | 81 |

### Example 9: Confocal Imaging of Lysosomes

MHT cells were treated with the Premo sensor and either DMSO or AZD8055 for 24 hours prior to the addition of 2 μM Compound No. 851810 by free uptake for 40 minutes, 2 hours, or 4 hours. The media was then replaced with fresh media containing LysoTracker (ThermoFisher) and cells were imaged 2 hours later. Confocal images were analyzed by counting the total number of lysosomes per cell and the number of lysosomes that colocalized with antisense oligonucleotide. Results represent the average of 6-10 images and are presented in Table 25.

TABLE 25
Colocalization of antisense oligonucleotide with lysosomes

| | | | | % of lysosomes co- | | |
|---|---|---|---|---|---|---|
| | Total number | | | localized with antisense | | |
| | lysosomes/cell | | | oligonucleotide | | |
| | 40 | 2 | 4 | 40 | 2 | 4 |
| Cell treatment | minutes | hours | hours | minutes | hours | hours |
| DMSO + 2 μM 851810 | 26 | 57 | 33 | 39 | 88 | 56 |
| AZD8055 + 2 μM 851810 | 65 | 185 | 122 | 53 | 80 | 56 |

### Example 10: Effect of COPB2 Inhibition on Antisense Activity in MHT Cells

COPB2 regulates the process of autophagosomes fusing with lysosomes. Inhibiting COPB2 can result in abortive autophagy and an increase in autophagosomes.

### Cell Treatment

MHT cells were plated at 10,000 cells/well and transfected with control siRNA or siRNA targeted to COPB2 using Lipofectamine RNAiMAX for 48 hours. An autophagy modulator or DMSO was then added for 24 hours. Fourteen hours after the addition of an autophagy modulator, a modified antisense or modified control oligonucleotide was added at 5 μM by free uptake for ten hours.

### RNA Analysis

RT-qPCR analysis was performed to detect RNA levels of Malat-1 as described in Example 1 above. In order to control for global effects of autophagy modulators on gene expression, Malat-1 RNA levels after treatment with siRNA, an antisense oligonucleotide, and an autophagy modulator were normalized to Malat-1 RNA levels in cells treated with the same siRNA, control Compound No. 141923, and the same autophagy modulator.

TABLE 26
Malat-1 RNA levels

| | Malat-1 level (% relative | |
|---|---|---|
| | to siRNA + 141923 + | |
| | autophagy modulator) | |
| Cell treatment | control siRNA | COPB2 siRNA |
| siRNA + 5 μM 399462 + DMSO | 36.3 | 14.2 |
| siRNA + 5 μM 399462 + AZD8055 | 10.1 | 17.5 |
| siRNA + 5 μM 399462 + Chloroquine | 14.1 | 10.3 |

### Example 11: Confocal Imaging of Lysosomes and Autophagic Vesicles

The Premo autophagy tandem sensor RFP-GFP-LC3B was used to visualize autophagy in MHT cells. LysoTracker was used to visualize lysosomes. MHT cells were treated with the Premo sensor and LysoTracker for 2 hours, then either DMSO or an autophagy modulator for 14 hours. Compound No. 851810 was then added at 2 μM by free uptake for 10 hours prior to cell imaging. Confocal images were analyzed by counting the total number of autophagic vesicles per cell and lysosomes per cell as well as the number of autophagic vesicles and lysosomes that colocalized with the antisense oligonucleotide of Compound No. 851810. Results represent the average of 4-6 images. Results are presented in Tables 27 and 28.

TABLE 27
Colocalization of antisense oligonucleotide with autophagic vesicles

| | | % of |
|---|---|---|
| | | autophagic |
| | Total | vesicles |
| | number | co-localized |
| | autophagic | oligonucleotide |
| Cell treatment | vesicles/cell | with antisense |
| DMSO + 851810 | 76.4 | 20 |
| AZD8055 + 851810 | 245.4 | 43 |
| Chloroquine + 851810 | 40.8 | 3.7 |

TABLE 28
Colocalization of antisense oligonucleotide with lysosomes

| | | % of |
|---|---|---|
| | | lysosomes |
| | Total | co-localized |
| | number | with |
| | lysosomes/ | antisense |
| Cell treatment | cell | oligonucleotide |
| DMSO + 851810 | 119 | 29 |
| AZD8055 + 851810 | 183 | 30 |

### Example 12: Confocal Imaging of Early Endosomes

CellLight early endosome (EE) marker was used to visualize early endosomes in MHT cells. MHT cells were treated with CellLight EE and either DMSO or 500 nM AZD8055 for 14 hours prior to the addition of Compound No. 851810 at 2 μM by free uptake for 10 hours. Confocal images were then obtained and analyzed by counting the total number of early endosomes per cell and the number of early endosomes that colocalized with the antisense oligonucleotide of Compound No. 851810. Results represent the average of 7-8 images. Results are presented in Table 29.

TABLE 29
Colocalization of antisense
oligonucleotide with autophagic vesicles

| | Total | % of EE |
|---|---|---|
| | number | co-localized |
| | early | with |
| | endosomes | antisense |
| Cell treatment | (EE)/cell | oligonucleotide |
| DMSO + 851810 | 53 | 24 |
| AZD8055 + 851810 | 55 | 34 |

### Example 13: Effects of Modulating Autophagy on Modified Oligonucleotide Activity In Vivo—AZD8055 Treatment

The following experiments were performed to determine if the effects of autophagy modulators on antisense activity in vitro can be observed in vivo. Groups of 3-4 8-week-old male C57bl/6J mice were administered 5 mg/kg control oligonucleotide 141923, or 5 mg/kg antisense oligonucleotide 399462. Following administration of modified oligonucleotide, mice were administered 1% DMSO or 5 mg/kg AZD8055 in 1% DMSO at three time points over 24 hours: at the time of administration of the modified oligonucleotide, and 12 and 22 hours after administration of the modified oligonucleotide. A group of control mice was administered PBS at time 0 and 1% DMSO at 0, 12, and 22 hours. Mice were sacrificed at 24 hours after administration of the modified oligonucleotide and tissues were collected for analysis.

Autophagy activation was confirmed in livers by determining suppression of mTOR kinase activity (pS6/S6 levels), determining an increased LC3B II/I ratio (a marker for the formation of autophagosomes), and determining p62 degradation (related to the clearance of autophagosomes). Protein analysis was carried out on liver tissue using standard procedures. All proteins were normalized to α-tubulin. Phospho-S6 Ribosomal Protein (Ser240/244) (2215S), and S6 Ribosomal Protein (2217S) antibodies were obtained from Cell Signaling Technology. The antibody against LC3B (ab48394) was purchased from Abcam. The antibody against alpha-tubulin (11224-1-AP) was purchased from Proteintech Group. Antibody against P62 (GP62-C) was purchased from Progen. Anti-rabbit (7074S) secondary antibody conjugated to HRP was from Cell Signaling Technology. IRDye® 800CW Donkey anti-Guinea Pig IgG (925-32411) secondary antibody was purchased from LI-COR, Inc. Results shown in Table 30 confirm that autophagy was activated in liver.

TABLE 30
Ratio of LC3B II/I and p62 levels after treatment

| Compound | | pS6/S6 | LC3B | |
|---|---|---|---|---|
| No. | Treatment | ratio | II/I ratio | P62 |
| PBS | DMSO | 1.0 | 1.0 | 1.0 |
| | AZD8055 | 0.2 | 2.3 | 0.5 |
| 141923 | DMSO | 1.1 | 1.0 | 1.4 |
| | AZD8055 | 0.2 | 2.0 | 0.7 |
| 399462 | DMSO | 0.7 | 1.3 | 1.1 |
| | AZD8055 | 0.2 | 2.2 | 0.5 |

MALAT-1 and Atg7 mRNA levels were measured by RT-qPCR analysis as described in Example 1 above. Atg7 mRNA levels are a marker of the induction of autophagy in treated mouse livers. Results are presented as percent change of RNA, relative to PBS control treated with DMSO, normalized to mouse cyclophilin A measured using mouse primer probe set Cyclophilin-A (forward sequence CACCATTGCTGACTGTGGAC, designated herein as SEQ ID NO: 38; reverse sequence ACTCTGCAATCCAGCTAGGC, designated herein as SEQ ID NO: 39; probe sequence TTCTGTAGCTCAGGAGAGCACCCCTCCACCCCATTTGCTCGCAGTATCCT, designated herein as SEQ ID NO: 40).

Results presented in Table 31 show that activation of autophagy in vivo significantly enhanced ASO activity as demonstrated by greater Malat1 RNA reduction in the livers of mice treated with AZD8055.

TABLE 31
Malat-1 and Atg7 mRNA levels in mouse liver

| | Malat-1 | Atg7 |
|---|---|---|
| | level (% | level (% |
| | relative to | relative to |
| | PBS/DMSO- | PBS/DMSO- |
| | treated | treated |
| Treatment | mice) | mice) |
| PBS + AZD8055 | 121 | 112 |
| 141923 + DMSO | 95 | 100 |
| 141923 + AZD8055 | 121 | 130 |
| 399462 + DMSO | 62 | 114 |
| 399462 + AZD8055 | 41 | 117 |

### Example 14: Effect of Modulating Autophagy by Fasting on Antisense Activity In Vivo

Groups of 4 8-week-old male C57bl/6J mice were administered 5 mg/kg control oligonucleotide 141923 or 5 mg/kg antisense oligonucleotide 399462. Following administration of modified oligonucleotide, groups of mice were either allowed free access to chow (control) or fasted overnight (16 hr fast). Mice were sacrificed at 16 hours after administration of the modified oligonucleotide and tissues were collected for analysis.

Autophagy activation was confirmed in livers by determining suppression of mTOR kinase activity (pS6/S6 levels), determining an increased LC3B II/I ratio (a marker for the formation of autophagosomes), and determining p62 degradation (related to the clearance of autophagosomes). Protein analysis was carried out on liver tissue using standard procedures. All proteins were normalized to α-tubulin. Phospho-S6 Ribosomal Protein (Ser240/244) (2215S), and S6 Ribosomal Protein (2217S) antibodies were obtained from Cell Signaling Technology. The antibody against LC3B (ab48394) was purchased from Abcam. The antibody against alpha-tubulin (11224-1-AP) was purchased from Proteintech Group. Antibody against P62 (GP62-C) was purchased from Progen. Anti-rabbit (7074S) secondary antibody conjugated to HRP was from Cell Signaling Technology. IRDye® 800CW Donkey anti-Guinea Pig IgG (925-32411) secondary antibody was purchased from LI-COR, Inc.

TABLE 32
Autophagy markers in vivo in fasted animals

| Compound | | pS6/S6 | LC3B | |
|---|---|---|---|---|
| No. | Treatment | ratio | II/I ratio | P62 |
| PBS | N.D. | 1.0 | 1.0 | 1.0 |
| | Fasting | 0.7 | 1.6 | 1.0 |
| 141923 | N.D. | 1.1 | 1.2 | 1.4 |
| | Fasting | 0.4 | 1.7 | 1.3 |
| 399462 | N.D. | 1.0 | 1.1 | 1.4 |
| | Fasting | 0.4 | 1.5 | 0.8 |

MALAT-1 and Atg7 mRNA levels were measured by RT-qPCR analysis as described in Example 1 above. Atg7 mRNA levels are a marker of the induction of autophagy in treated mouse livers. Results are presented in Table 33 relative to mice treated with PBS and allowed normal access to chow. These results demonstrate that induction of autophagy with rapamycin leads to a significant enhancement of modified oligonucleotide activity in vivo.

TABLE 33
Malat-1 and Atg7 mRNA levels in mouse
liver after treatment with MOE gapmers

| Compound | | MALAT1 | Atg7 |
|---|---|---|---|
| No. | Treatment | (% PBS) | (% PBS) |
| PBS | Normal Chow | 100 | 1.0 |
| | Fasting | 100 | 1.49 |
| 141923 | Normal Chow | 119 | 1.0 |
| | Fasting | 99 | 1.39 |
| 399462 | Normal Chow | 87 | 0.74 |
| | Fasting | 66 | 1.08 |

### Example 15: Effects of Modulating Antisense Activity In Vivo with Autophagy Activator Rapamycin

Groups of 4 8-week-old male C57bl/6J mice were administered 5 mg/kg control oligonucleotide 141923 or 5 mg/kg antisense oligonucleotide 399462. Following administration of modified oligonucleotide, mice were administered 1% DMSO or 5 mg/kg rapamycin in 1% DMSO to activate autophagy at three time points over 24 hours: at the time of administration of the modified oligonucleotide, and 12 and 22 hours after administration of the modified oligonucleotide. A group of control mice was administered PBS at time 0 and 1% DMSO at 0, 12, and 22 hours. Mice were sacrificed at 24 hours after administration of the modified oligonucleotide and tissues were collected for analysis.

Autophagy activation was confirmed in livers by determining suppression of mTOR kinase activity (pS6/S6 levels), determining an increased LC3B II/I ratio (a marker for the formation of autophagosomes), and determining p62 degradation (related to the clearance of autophagosomes). Protein analysis was carried out on liver tissue using standard procedures. All proteins were normalized to α-tubulin. Phospho-S6 Ribosomal Protein (Ser240/244) (2215S), and S6 Ribosomal Protein (2217S) antibodies were obtained from Cell Signaling Technology. The antibody against LC3B (ab48394) was purchased from Abcam. The antibody against alpha-tubulin (11224-1-AP) was purchased from Proteintech Group. Antibody against P62 (GP62-C) was purchased from Progen. Anti-rabbit (7074S) secondary antibody conjugated to HRP was from Cell Signaling Technology. IRDye® 800CW Donkey anti-Guinea Pig IgG (925-32411) secondary antibody was purchased from LI-COR, Inc. Results presented in Table 34 demonstrate that autophagy was activated in liver.

TABLE 34
Autophagy markers in vivo in fasted animals

| Compound | | pS6/S6 | LC3B | |
|---|---|---|---|---|
| No. | Treatment | ratio | II/I ratio | P62 |
| PBS | DMSO | 1.0 | 1.0 | 1.0 |
| | Rapamycin | 0.7 | 1.3 | 0.8 |
| 141923 | DMSO | 0.9 | 1.0 | 1.0 |
| | Rapamycin | 0.6 | 1.3 | 1.2 |
| 399462 | DMSO | 0.9 | 1.1 | 1.1 |
| | Rapamycin | 0.7 | 1.3 | 1.0 |

MALAT-1 and Atg7 mRNA levels were measured by RT-qPCR analysis as described in Example 1 above. Atg7 mRNA levels are a marker of the induction of autophagy in treated mouse livers. Results are presented relative to mice treated with PBS and 1% DMSO. Results presented in Table 35 demonstrate that autophagy activation enhances antisense activity in vivo.

TABLE 35
Malat-1 and Atg7 mRNA levels in mouse
liver after treatment with MOE gapmers

| | Malat-1 | Atg7 |
|---|---|---|
| | level (% | level (% |
| | relative to | relative to |
| | PBS/DMSO | PBS/DMSO |
| Treatment | mice) | mice) |
| PBS + rapamycin | 89 | 111 |
| 141923 (control) + DMSO | 105 | 96 |
| 141923 (control) + rapamycin | 108 | 111 |
| 399462 + DMSO | 59 | 93 |
| 399462 + rapamycin | 39 | 105 |

### Example 16: Effect of Modulating Autophagy by Fasting on Modified Oligonucleotide Activity In Vivo—Ketogenic Diet

To activate autophagy, groups of 3-4 male C57BL/6J mice were subjected to a 5-week ketogenic diet which has been shown to inhibit mTORC1 activity and activate autophagy in mice. Mice were treated with either PBS, 5 mg/kg of Compound No. 549148, added as a control modified oligonucleotide, or 5 mg/kg of Compound No. 556089 that targets mouse MALAT1 (total of 1 administration of modified oligonucleotide) at the end of the 5-weeks on the ketogenic diet. Mice were sacrificed 16 hours post modified oligonucleotide administration and liver tissue was collected to examine the effects of autophagy induction on modified oligonucleotide activity.

Autophagy activation was confirmed in livers by determining suppression of mTOR kinase activity (pS6/S6 levels), determining an increased LC3B II/I ratio (a marker for the formation of autophagosomes), and determining p62 degradation (related to the clearance of autophagosomes). Protein analysis was carried out on liver tissue using standard procedures. All proteins were normalized to α-tubulin. Phospho-S6 Ribosomal Protein (Ser240/244) (2215S), and S6 Ribosomal Protein (2217S) antibodies were obtained from Cell Signaling Technology. The antibody against LC3B (ab48394) was purchased from Abcam. The antibody against alpha-tubulin (11224-1-AP) was purchased from Proteintech Group. Antibody against P62 (GP62-C) was purchased from Progen. Anti-rabbit (7074S) secondary antibody conjugated to HRP was from Cell Signaling Technology. IRDye® 800CW Donkey anti-Guinea Pig IgG (925-32411) secondary antibody was purchased from LI-COR, Inc. Results presented in Table 36 demonstrate that autophagy was activated in liver.

TABLE 36
Autophagy markers in vivo in animals on ketogenic diet

| Compound | | pS6/S6 | LC3B | |
|---|---|---|---|---|
| No. | Treatment | ratio | II/I ratio | P62 |
| PBS | N.D. | 1.0 | 1.0 | 1.0 |
| | Ketogenic | 0.3 | 1.6 | 0.5 |
| 549148 | N.D. | 0.8 | 1.4 | 1.0 |
| (control) | Ketogenic | 0.3 | 2.3 | 0.5 |
| 556089 | N.D. | 2.1 | 1.6 | 1.0 |
| | Ketogenic | 0.6 | 2.5 | 0.6 |

RNA was extracted from liver tissue for real-time RTPCR analysis of MALAT1 RNA expression. Primer probe set 15877 was used to measure mouse MALAT1 RNA levels. Results are presented as percent change of RNA, relative to PBS control, normalized to mouse cyclophilin A measured using mouse primer probe set Cyclophilin-A.

Results presented in Table 37 show that diet induced activation of autophagy in vivo significantly enhanced antisense activity as demonstrated by greater Malat1 RNA reduction in the livers of mice on a ketogenic diet.

TABLE 37
MALAT1 Antisense activity in vivo
in mice on ketogenic diet

| Compound | | MALAT1 |
|---|---|---|
| No. | Treatment | (% PBS) |
| PBS | N.D. | 100 |
| | Ketogenic | 101 |
| 549148 | N.D. | 102 |
| (control) | Ketogenic | 91 |
| 556089 | N.D. | 40 |
| | Ketogenic | 17 |

### Example 17: Time Course of Antisense Activity In Vivo with Ketogenic Diet Induced Autophagy

To activate autophagy, groups of 4 male C57BL/6J mice were subjected to a 5-week ketogenic diet which has been shown to inhibit mTORC1 activity and activate autophagy in mice. Mice were treated with either PBS, 5 mg/kg of Compound No. 549148, added as a control modified oligonucleotide, or 5 mg/kg of Compound No. 556089 that targets mouse MALAT1 (total of 1 administration of modified oligonucleotide) at the end of the 5-weeks on the ketogenic diet. Mice were sacrificed 24, 48 and 72 hours post modified oligonucleotide administration and liver tissue was collected to examine the effects of autophagy induction on modified oligonucleotide activity.

RNA was extracted from liver tissue for real-time RTPCR analysis of MALAT1 RNA expression. Primer probe set 15877 was used to measure mouse MALAT1 RNA levels. Results are presented as percent change of RNA, relative to PBS control, normalized to mouse cyclophilin A measured using mouse primer probe set Cyclophilin-A.

As evidenced by the results presented in Table 38, level of knockdown of MALAT1 does not change between 24 and 72 hours on normal chow. However, diet induced activation of autophagy in vivo significantly enhanced ASO activity as demonstrated by greater Malat1 RNA reduction in the livers of mice on a ketogenic diet, with cET gapmers. Knockdown was found to be further increased at 72 hours compared to 24 hours on the ketogenic diet.

TABLE 38
MALAT1 3-10-3 cET gapmer activity
in vivo on ketogenic diet

| Compound | | Time of | MALAT1 |
|---|---|---|---|
| No. | Treatment | sac (hrs) | (% PBS) |
| PBS | normal chow | 72 | 100 |
| | ketogenic | 72 | 105 |
| 549148 | normal chow | 24 | 77 |
| (control) | ketogenic | 24 | 122 |
| | normal chow | 72 | 86 |
| | ketogenic | 72 | 111 |
| 556089 | normal chow | 24 | 30 |
| | ketogenic | 24 | 19 |
| | normal chow | 48 | 34 |
| | ketogenic | 48 | 13 |
| | normal chow | 72 | 33 |
| | ketogenic | 72 | 10 |

### Example 18: Effect of Modulation of Autophagy on Antisense Activity in Human Fibroblasts

### Cell Treatment

Human fibroblasts were plated at 8000 cells/well and treated with DMSO (control) or an autophagy modulator (AZD8055) for 24 hours, at the concentrations indicated in Example 1. Fourteen hours after the addition of an autophagy modulator, Compound No. 556089 was added at 10 μM by free uptake for ten hours, as indicated in the tables below.

### RNA Analysis

RT-qPCR analysis was performed to detect RNA levels of Malat-1 using primer probe set RTS2736. Results were normalized to cyclophilin A levels.

Induction of autophagy significantly enhanced activity of modified oligonucleotides (cET gapmers) targeting MALAT1 in human fibroblasts, as evidenced by the results presented in Table 39.

TABLE 39
Malat-1 RNA levels in human
fibroblasts with cET gapmer

| | MALAT1 |
|---|---|
| Treatment | (% PBS) |
| 556089 + DMSO | 68 |
| 556089 + AZD8055 | 50 |

### Example 19: Effect of Modulation of Autophagy on Antisense Activity in Primary Mouse Hepatocytes

### Cell Treatment

Primary mouse hepatocytes were plated at 10,000 cells/well and treated with DMSO (control) or an autophagy modulator (AZD8055) for 24 hours, at the concentrations indicated in Example 1. Fourteen hours after the addition of an autophagy modulator, Compound No. 399462 was added at dose response 0, 12, 37, 111, 333, 1000, and 3000 nM by free uptake for ten hours, as indicated in the tables below. In a separate experiment, primary mouse hepatocytes, plated at xxxcells/well were treated with DMSO (control) or an autophagy modulator (AZD8055) for 24 hours, at the concentrations indicated in Example 1. Fourteen hours after the addition of an autophagy modulator, Compound No. 841226 (a 5-10-5 MOE gapmer that targets mouse MALAT1 and has the sequence (from 5′ to 3′): CCAGGCTGGTTATGACTCAG, with a Cy3 (IDT 5Cy3 fluorophore) conjugate on the 5′ end of the sequence and a THA-C6-GalNAc hydroxyproline PO conjugate at the 3′ end of the sequence, designated herein as SEQ ID No. 41. Every internucleoside linkage of Compound No. 841226 is a phosphorothioate internucleoside linkage. All of the cytosines had a methyl at the 5 position.)

### RNA Analysis

RT-qPCR analysis was performed to detect RNA levels of Malat-1 using primer probe set previously described in Example 1. Results were normalized to cyclophilin A levels.

Induction of autophagy significantly enhanced activity of modified oligonucleotides (both unconjugated and conjugated MOE gapmers) targeting MALAT1 in mouse primary hepatocytes, as evidenced by the examples presented in Tables 40 and 41.

TABLE 40
Malat-1 RNA levels in mouse primary hepatocytes
with unconjugated MOE gapmer

| | % UTC | |
|---|---|---|
| Concentration | (399462 + | 399462 + |
| (nM) | DMSO) | AZD8055 |
| PBS | 100 | 100 |
| 12 nM | 76 | 66 |
| 37 nM | 65 | 53 |
| 111 nM | 63 | 53 |
| 333 nM | 55 | 44 |
| 1000 nM | 47 | 40 |
| 3000 nM | 43 | 34 |

TABLE 41
Malat-1 RNA levels in mouse primary heptocytes
with Galnac conjugated MOE gapmer

| | % UTC | % UTC |
|---|---|---|
| Concentration | (841226 + | (841226 + |
| (nM) | DMSO) | AZD8055) |
| PBS | 100 | 100 |
| 12 nM | 80 | 48 |
| 37 nM | 59 | 38 |
| 111 nM | 59 | 27 |
| 333 nM | 43 | 22 |
| 1000 nM | 38 | 22 |
| 3000 nM | 41 | 24 |

### Example 20: Effect of Modulation of Autophagy on Antisense Activity in Mouse Embryonic Fibroblasts

### Cell Treatment

Mouse embryonic fibroblasts (MEFs) were plated at 8000 cells/well and treated with DMSO (control) or an autophagy modulator (AZD8055) for 24 hours, at the concentrations indicated in Example 1. Fourteen hours after the addition of an autophagy modulator, Compound No. 353382 was added at 3000 nM, 1000 nM and 500 nM by free uptake for ten hours, as indicated in the tables below.

In a separate experiment, mouse embryonic fibroblasts (MEFs) were plated at 8000 cells/well and treated with DMSO (control) or an autophagy modulator (AZD8055) for 24 hours, at the concentrations indicated in Example 1. Fourteen hours after the addition of an autophagy modulator, Compound No. 399462 was added at 1000 nM, 500 nM and 250 nM by free uptake for ten hours, as indicated in the tables below.

### RNA Analysis

RT-qPCR analysis was performed to detect mRNA levels of SRB1 using primer probe set 15299, and to detect MALAT1 levels using primer probe set 15877. Results were normalized to cyclophilin A levels and are presented as relative to untreated control (% UTC).

Induction of autophagy significantly enhanced activity of modified oligonucleotides against multiple targets in mouse embryonic fibroblasts, as evidenced by the results presented in Tables 42 and 43.

TABLE 42
SRB1 RNA levels in MEFs with MOE gapmer

| | % UTC | % UTC |
|---|---|---|
| Concentration | (353382 + | (353382 + |
| (nM) | DMSO) | AZD8055) |
| 3000 nM | 65 | 54 |
| 1000 nM | 74 | 64 |
| 500 nM | 72 | 68 |

TABLE 43
MALAT1 RNA levels in MEFs with MOE gapmer

| | % UTC | % UTC |
|---|---|---|
| Concentration | (399462 + | (399462 + |
| (nM) | DMSO) | AZD8055) |
| 1000 nM | 88 | 73 |
| 500 nM | 89 | 67 |
| 250 nM | 102 | 75 |

### Example 21: Effect of Modulation of Autophagy on Antisense Activity in Human Lung Epithelial Cells

### Cell Treatment

H460 cells were plated at 7500 cells/well and treated with DMSO (control) or an autophagy modulator (AZD8055) for 24 hours, at the concentrations indicated in Example 1. Fourteen hours after the addition of an autophagy modulator, Compound No. 395254 was added at 1000 nM by free uptake for ten hours.

### RNA Analysis

RT-qPCR analysis was performed to detect mRNA levels of MALAT1 using primer probe set RTS2736. Results were normalized to cyclophilin A levels and are presented as relative to untreated control (% UTC).

Induction of autophagy significantly enhanced activity of modified oligonucleotides targeting MALAT1 in H460 lung epithelial carcinoma cells, as evidenced by the results presented in Table 44.

TABLE 44
MALAT1 RNA levels in H460 cells with MOE gapmer

| | % UTC | % UTC |
|---|---|---|
| Concentration | (395254 + | (395254 + |
| (nM) | DMSO) | AZD8055) |
| 1000 nM | 100 | 66 |

### Example 22: Effect of Modulation of Autophagy on Antisense Activity in Human Brain Neuroglioma Cells

### Cell Treatment

H4 cells (a cell line derived from a human brain neuroglioma) were plated at 7500 cells/well and treated with DMSO (control) or an autophagy modulator (AZD8055) for 24 hours, at the concentrations indicated in Example 1. Fourteen hours after the addition of an autophagy modulator, Compound No. 395254 was added at 5000 nM by free uptake for ten hours.

### RNA Analysis

RT-qPCR analysis was performed to detect mRNA levels of MALAT1 using primer probe set RTS2736. Results were normalized to cyclophilin A levels and are presented as relative to untreated control (% UTC).

Induction of autophagy significantly enhanced activity of modified oligonucleotides targeting MALAT1 in H4 brain neuroglioma cells, as evidenced by the results presented in Table 45.

TABLE 45
MALAT1 RNA levels in H4 cells with MOE gapmer

| | % UTC | % UTC |
|---|---|---|
| Concentration | (395254 + | (395254 + |
| (nM) | DMSO) | AZD8055) |
| 5000 nM | 71 | 25 |

### Example 23: Effect of Modulation of Autophagy on Antisense Activity In Vitro in HT1080 Cells (Fibrosarcoma Cells), Multiple Dose

### Cell Treatment

HT1080 cells were plated at 7500 cells/well and treated with DMSO (control) or an autophagy modulator (AZD8055) for 24 hours, at the concentrations indicated in Example 1. Fourteen hours after the addition of an autophagy modulator, various modified oligonucleotides were added per table 45. All the compounds described in Table 46 have uniform phosphorothioate internucleoside linkages. All of the cytosines had a methyl at the 5 position. All compounds were added to cells at 5000 nM.

TABLE 46
Modified Oligonucleotides

| | | | Target to | |
|---|---|---|---|---|
| | | | which it | |
| | | | is 100% | SEQ |
| Compound | | | com- | ID |
| No. | Sequence | chemistry | plementary | NO |
| 481549 | GAAATTCATTCTTCCA | 3-10-3 cET | STAT3 | 42 |
| 116847 | CTGCTAGCCTCTGGAT | 5-10-5 MOE | PTEN | 30 |
| | TTGA | | | |
| 597453 | GTATGCTGTGGCTTTT | 3-10-3 cET | c-myc | 43 |

### RNA Analysis

RT-qPCR analysis was performed to detect RNA levels of STAT3 using primer probe set RTS96 (forward sequence AATGGCTAAGTGAAGATGACAATCAT, designated herein as SEQ ID NO: 44; reverse sequence TGCACATATCATTACACCAGTTCGT, designated herein as SEQ ID NO: 45; probe sequence TTGCAGCAATTCACTGTAAAGCTGGAAAGG, designated herein as SEQ ID NO: 46). PTEN RNA levels were detected using Thermofisher primer probe set Hs00374280 ml. c-myc RNA levels were detected using Thermofisher primer-probe set Hs00153408_ml. Results were normalized to cyclophilin A levels (measured using HTS3936) and are presented as relative to untreated control (% UTC).

Induction of autophagy significantly enhanced activity of modified oligonucleotides targeting STAT3, PTEN and c-myc in HT1080 cells, as evidenced by results shown in Tables 47-49.

TABLE 47
STAT3 RNA levels in HT1080 cells with cET gapmer

| | % UTC | % UTC |
|---|---|---|
| Concentration | (481549 + | (481549 + |
| (nM) | DMSO) | AZD8055) |
| 5000 nM | 91 | 69 |

TABLE 48
PTEN RNA levels in HT1080 cells with MOE gapmer

| | % UTC | % UTC |
|---|---|---|
| Concentration | (116847 + | (116847 + |
| (nM) | DMSO) | AZD8055) |
| 5000 nM | 88 | 68 |

TABLE 49
c-myc RNA levels in HT1080 cells with MOE gapmer

| | % UTC | % UTC |
|---|---|---|
| Concentration | (597453 + | (597453 + |
| (nM) | DMSO) | AZD8055) |
| 5000 nM | 110 | 82 |

1. A method comprising
activating autophagy of a cell; and
contacting the cell with an antisense compound comprising an antisense oligonucleotide, wherein the nucleobase sequence of the antisense oligonucleotide is complementary to a target nucleic acid.

2. The method of claim 1, wherein activating autophagy does not comprise contacting the cell with a nucleic acid delivery vehicle.

3. The method of claim 1 or 2, wherein the cell is not contacted with a nucleic acid delivery vehicle.

4. The method of any of claims 1-3, wherein an amount or expression of the target nucleic acid in the cell is modified.

5. The method of claim 4, wherein the expression or amount of the target nucleic acid is modified to a greater extent than in the absence of activating autophagy.

6. The method of claim 1, wherein the nucleobase sequence of the antisense oligonucleotide is at least 80%, at least 85%, at least 90%, at least 95%, or at least 100% complementary to the target nucleic acid.

7. The method of claim 1, wherein the antisense oligonucleotide is a modified oligonucleotide.

8. The method of claim 7, wherein the modified oligonucleotide is a gapmer.

9. The method of claim 7, wherein the antisense oligonucleotide comprises at least one modified internucleoside linkage.

10. The method of claim 9, wherein the at least one modified internucleoside linkage is a phosphorothioate internucleoside linkage.

11. The method of claim 1, wherein the antisense oligonucleotide comprises at least one modified sugar moiety.

12. The method of claim 11, wherein the at least one modified sugar moiety comprises a 2′-MOE, 2′-O-methyl, cEt, or LNA modification.

13. The method of claim 1, wherein the antisense compound is single-stranded.

14. The method of claim 1, comprising activating autophagy in a subject and administering the antisense compound to the subject.

15. The method of claim 1, wherein activating autophagy comprises contacting the cell with an autophagy modulator.

16. The method of claim 15, wherein the autophagy modulator increases autophagosome formation in the cell relative to autophagosome formation in absence of the autophagy modulator.

17. The method of claim 15, wherein the autophagy modulator increases autophagosome nucleation and/or autophagosome elongation relative to autophagosome nucleation and/or autophagosome elongation in absence of the autophagy modulator.

18. The method of claim 15, wherein the autophagy modulator increases the number of autophagic vesicles in the cell relative to the number of autophagic vesicles in the absence of the autophagy modulator.

19. The method of claim 15, wherein the autophagy modulator increases the number of autophagosomes in the cell relative to the number of autophagosomes in the cell in the absence of the autophagy modulator.

20. The method of claim 15, wherein the autophagy modulator is an mTor inhibitor.

21. The method of claim 15, wherein the autophagy modulator is rapamycin or a rapalog.

22. The method of claim 15, wherein the autophagy modulator is AZD8055.

23. The method of claim 15, wherein the autophagy modulator blocks fusion of autophagosomes to lysosomes.

24. The method of claim 15, wherein the autophagy modulator is Vinblastine.

25. The method of claim 15, wherein the autophagy modulator is Bafilomycin A1.

26. A composition comprising an autophagy modulator and an antisense compound.

27. The composition of claim 26, wherein the autophagy modulator is rapamycin or a rapalog.

28. The composition of claim 26, wherein the autophagy modulator is AZD8055.

29. The composition of claim 26, wherein the autophagy modulator comprises a single stranded, modified oligonucleotide and an autophagy modulator selected from rapamycin, a rapalog, and AZD8055.
